(19) United States
(12) Patent Application Publication
(10) Pub. No.: US 2024/0335453 A1
(43) Pub. Date: Oct. 10, 2024

(54) METALLO-ß-LACTAMASE INHIBITORS

(71) Applicants: University of KwaZulu-Natal, Westville (ZA); University of Zululand, Richards Bay (ZA)

(72) Inventors: Thavendran Govender, Richards Bay (ZA); Tricia Naicker, Chatsworth (ZA); Per I Arvidsson, Westville (ZA); Hendrik G Kruger, Westville (ZA); Byron Peters, Westville (ZA)

(21) Appl. No.: 18/292,703

(22) PCT Filed: Jul. 21, 2022
(86) PCT No.: PCT/IB2022/056748

§ 371 Date: Jan. 26, 2024

(30) Foreign Application Priority Data
Jul. 28, 2021 (ZA) 2021/05326

(51) Int. Cl. A61K 31/546; A61K 31/407; A61K 31/431; A61P 31/04

(52) U.S. Cl. CPC A61K 31/546; A61K 31/407; A61K 31/431; A61P 31/04

(57) ABSTRACT

A metallo-β-lactamase (MBL) inhibitor of general formula (I) C-A-L (I) in which: C is a functionalised aza-cycloalkane zinc chelating moiety, L is a β-lactam moiety which may be functionalised, and A is a C₁-C₁₀ linear or cyclic linker, which may be functionalised, is provided.

### FIELD OF THE INVENTION

THIS INVENTION relates to inhibitors of metallo-β-lactamases (MBLs).

### BACKGROUND TO THE INVENTION

β-LACTAM ANTIBIOTICS are some of the most widely employed antibiotics and their successful use in the treatment of infectious diseases is well documented. However bacterial resistance to all known β-lactam antibiotics is escalating globally and the resultant loss of treatment options for infectious diseases is a threat to public health. One of the major reasons for bacterial resistance to β-lactam antibiotics is the production, by the bacteria, of metallo-β-lactamase enzymes. These enzymes hydrolyse the β-lactam ring of the antibiotic thereby inactivating the molecule. Metallo-β-lactamases (MBLs) are produced for example by pathogens such as Enterobacteriaceae and other gram-negative bacteria.

MBL-producing Enterobacteriaceae are the least susceptible to antibiotics among carbapenem-resistant Enterobacteriaceae (CRE) (Meletis G. Carbapenem resistance: overview of the problem and future perspectives. Ther Adv Infect Dis. 2016; 3(1):15-21.). MBLs have been identified worldwide and occur across Africa (Gupta N, Limbago B M, Patel J B et al. Carbapenem-resistant Enterobacteriaceae: epidemiology and prevention. Clinical infectious diseases 2011; 53: 60-7). The carbapenem class of β-lactam antibiotics had been held in reserve as agents of last resort due to their resistance to the action of MBLs, however enzymes capable of hydrolyzing carbapenem antibiotics are emerging in Gram-negative bacteria leading to the rapid resistance to this class of drug. In previous studies on MBL inhibitors a number of compounds showed good in vitro activity but were rejected in clinical trials because of cytotoxic effects, poor pharmacological properties or unfavorable effects on human metallo-enzymes (Tooke C L, Hinchliffe P, Bragginton E C, Colenso C K, Hirvonen V H A, Takebayashi Y, et al. β-Lactamases and β-Lactamase Inhibitors in the 21st Century. Journal of Molecular Biology. 2019; 431(18):3472-500.).

There are currently essentially no clinical inhibitors available for the inhibition of MBLs and the development of inhibitors of MBLs is currently receiving worldwide attention (Palacios A R, Rossi M-A, Mahler G S, Vila A J. Metallo-β-Lactamase Inhibitors Inspired on Snapshots from the Catalytic Mechanism. Biomolecules. 2020; 10(6):854.).

Carbapenem hydrolyzing enzymes also known as carbapenemases belong to either Ambler class A, B or D based on the reactive site of the enzyme. Classes A and D are serine carbapenemases such as KPC-2, OXA-48 and SME-1 which attack the β-lactam ring covalently and class B are MBLs such as NDM-1, VIM-1 and IMP-1 which employ Zn⁺⁺ ions to activate nucleophilic water molecules to open the β-lactam ring of the antibiotic.

A number of β-lactamase inhibitor combinations such as amoxicillin/clavulanate, ampicillin/sulbactam, piperacillin/tazobactam and ceftazidime and avibactam have been used in clinical practice, and new inhibitors are currently undergoing clinical trials. Cyclic boronates i.e Taniborbactam ((NCT03840148) and QPX7728 (NCT04380207), have displayed good MBL inhibitor activity towards type B β-lactamases. Both compounds are currently in phase 11 and phase I clinical trials, respectively, however their mechanism of inhibition is not yet completely understood. It has also been found by Docquier et al (J Antimicrobe Chemother. 2003 February; 51(2):257-66) that VIM-2 MBL is susceptible to inactivation by chelators, indicating that the zinc cations of the enzyme are probably loosely bound.

A few classes of non-toxic MBL inhibitors such as Aspergillomarasmine A (King A M, Reid-Yu S A, Wang W et al. Aspergillomarasmine A overcomes metallo-[bgr]-lactamase antibiotic resistance. Nature 2014; 510: 503-6) which is a rapid and potent inhibitor of the NDM-1 and VIM-2 MBLs, have been identified. This agent removes Zn²⁺ ions by a loss of 1.8 Zn equivalents in NDM-1, thus acting as an in vitro chelator that interacts with subclass B1 MBLs. NOTA, DOTA and DPA (Somboro A M, Tiwari D, Bester L A et al. NOTA: a potent metallo-β-lactamase inhibitor. Journal of Antimicrobial Chemotherapy 2015; 70: 1594-6; Azumah R, Dutta J, Somboro A M et al. In vitro evaluation of metal chelators as potential metallo-β-lactamase inhibitors. Journal of applied microbiology 2016) have also been reported to inhibit MBLs and, in a fairly recent PCT publication, Rongved et al. have reported new MBL inhibitors (Rongved P, Åstrand O A H, Bayer A et al. Inhibitors of Metallo-Beta-Lactamase (MBL) Comprising a Zinc Chelating Moiety. WO 2015/049546). The in vitro effects and some in vivo effects of these inhibitors have also been reported (King et al; von Nussbaum F, Schiffer G. Aspergillomarasmine A, an Inhibitor of Bacterial Metallo-β-Lactamases Conferring blaNDM and blaVIM Resistance. Angewandte Chemie International Edition 2014; 53: 11696-8; Rongved et al).

The use of “stand-alone” zinc chelators such as 1,4,7-triazacyclononane-1,4,7-triaceticacid (NOTA), 1,4,7,10-tetraazacyclononane-1,4,7,10-tetraaceticacid (DOTA) and dipicoylamine (DPA) which are all metal chelating agents as potent inhibitors of MBL producing Enterobacteriaceae and which have no toxic nor hemolytic effects at effective concentrations in vitro has also been reported (Somboro et al; Azumah et al). However, such stand-alone chelators were found not to possess the necessary pharmacokinetic profile to enable them to be used in combination with a β-lactam antibiotic.

Accordingly, the prime objective of antimicrobial chemotherapy (Craig W A, Welling P. Protein binding of antimicrobials: clinical pharmacokinetic and therapeutic implications. Clinical pharmacokinetics 1977; 2: 252-68) is to develop MBL inhibitors which can reach and maintain effective concentrations for treating infections. One of the factors that influence the physiological and pharmacological properties of MBL inhibitors is the interaction of these agents with serum and tissue proteins. This makes in vitro experimental assays that ascertain these properties very important as they may provide information on the mode of action in vivo of the drug (Arhin F F, McKay G A, Beaulieu S et al. Impact of human serum albumin on oritavancin in vitro activity against Staphylococcus aureus. Diagnostic microbiology and infectious disease 2009; 65: 207-10; McKay G A, Beaulieu S, Sarmiento I et al. Impact of human serum albumin on oritavancin in vitro activity against enterococci. Antimicrobial agents and chemotherapy 2009; 53: 2687-9).

The inventors have now developed a new class of MBL inhibitors with improved pharmacological properties by the attachment of a beta-lactam moiety to a bifunctional cyclic zinc chelator to afford efficient combination therapy when administered with a beta-lactam antibiotic. It is well documented in several studies that simple metal chelators undergo rapid renal clearance (Prata M I, Santos A c Fau-Geraldes C F, Geraldes C f Fau-de Lima J J, de Lima J J. Characterisation of 67Ga³⁺ complexes of triaza macrocyclic ligands: biodistribution and clearance studies. Nucl Med Biol. 1999; 26 (6):707-10; Reddy N, Shungube M, Arvidsson P I, Baijnath S, Kruger H G, Govender T, et al. A 2018-2019 patent review of metallo beta-lactamase inhibitors. Expert Opinion on Therapeutic Patents. 2020:1-15.) which would not match that of the accompanying beta-lactam drug.

### SUMMARY OF THE INVENTION

ACCORDING TO ONE ASPECT OF THE INVENTION there is provided a metallo-β-lactamase (MBL) inhibitor of general formula (I)

C-A-L (I)
in which:
C is a functionalised aza-cycloalkane zinc chelating moiety,
L is a β-lactam moiety which may be functionalised, and
A is a C₁-C₁₀ linear or cyclic linker, which may be functionalised.

The functionalised aza-cycloalkane zinc chelating moiety C may be a functionalised aza-cyclononane moiety.

Functionalised aza-cycloalkane zinc chelating moieties C useful in the invention typically have ring nitrogen atoms and carboxylic acid substituents groups, or ring nitrogen atoms and pyridyl substituents, and these zinc chelating moieties are also referred to as “bifunctional zinc chelating moieties” to indicate the two types of coordinating sub-structures namely the ring nitrogen atoms and the carboxylic or pyridyl groups which complex with the zinc.

Examples of some bifunctional cyclic chelators useful in the invention are set out in Scheme 1.

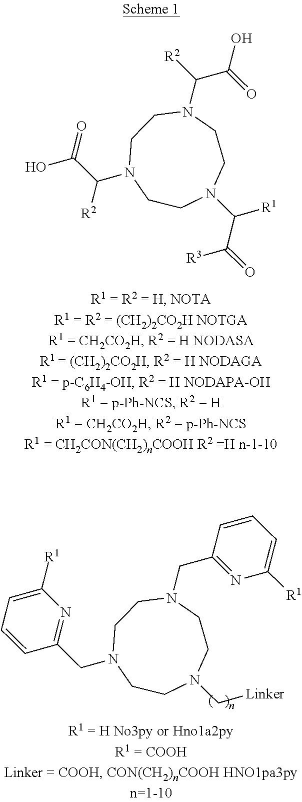

The functionalised aza-cycloalkane zinc chelating moiety C may thus be a substituted 1,4,7-triazacyclononane moiety.

The substituted 1,4,7-triazacyclononane moiety may be selected from the group consisting of a 1,4,7-triazacyclononane-1,4,7-triacetic acid (NOTA) moiety, a 1,4,7-triazacyclononane-1,4,7-triglutaric acid (NOTGA) moiety, a 1,4,7-triazacyclononane-1-succinic acid-4,7-diacetic acid (NODASA) moiety, a 1,4,7-triazacyclononane-1-glutaric acid-4,7-diacetic acid (NODAGA) moiety, a 1,4,7-triazacyclononane-1,4-diacetic acid-7-p-hydroxyphenyl-acetic acid (NODAPA) moiety, a 1,4,7-tris(2-pyridylmethyl)-1,4,7-triazacyclononane moiety, a 4,7-di(2-pyridylmethyl)-1,4,7-triazacyclononane-1-acetic acid, and a 1-[(5-carboxy-2-methylpyridyl)]-4,7-bis(2-methylpyridyl)-1,4,7-triazacyclononane moiety. It is however to be appreciated that the substituted 1,4,7-triazacyclononane moiety may not necessarily be limited to the moieties from the aforementioned group.

The linker A may be linked or connected to a ring nitrogen atom of the functionalised aza-cycloalkane zinc chelating moiety C. It may for example be formed by an aza-Michael addition of a nitrogen atom of the functionalised aza-cycloalkane zinc chelating moiety C to an α,β-unsaturated carbonyl compound such as an α,β-unsaturated ester. The ester may for example be an ester of an α,β-unsaturated C₄-C₁₀ mono- or diacid.

The linker A may thus be connected to a nitrogen atom of the substituted 1,4,7-triazacyclononane moiety.

The linker A may be a moiety selected from the group consisting of saturated monoacid moieties, unsaturated monoacid moieties, saturated diacid moieties, unsaturated diacid moieties and esters thereof.

The linker A may for example be derived from saturated or unsaturated monoacids or saturated or unsaturated diacids such as acetic acid, glutaric acid, maleic acid, fumaric acid, succinic acid or p-hydroxyphenyl acetic acid or the esters thereof.

In one embodiment of the invention, the linker A is thus a moiety selected from the group consisting of an acetic acid moiety, a glutaric acid moiety, a maleic acid moiety, a fumaric acid moiety, a succinic acid moiety, a p-hydroxyphenyl acetic acid moiety and the esters thereof.

The β-lactam moieties L of the metallo-β-lactamase (MBL) inhibitors of the invention may be derived from but not limited to cephalosporins, penems or carbapenems. Thus, in one embodiment of the invention, the β-lactam moiety L is selected from the group consisting of functionalised cephalosporin moieties, penem moieties and carbapenem moieties.

Examples of cephalosporins, penems or carbapenems from which the moieties may be derived or selected are set out in Scheme 2.

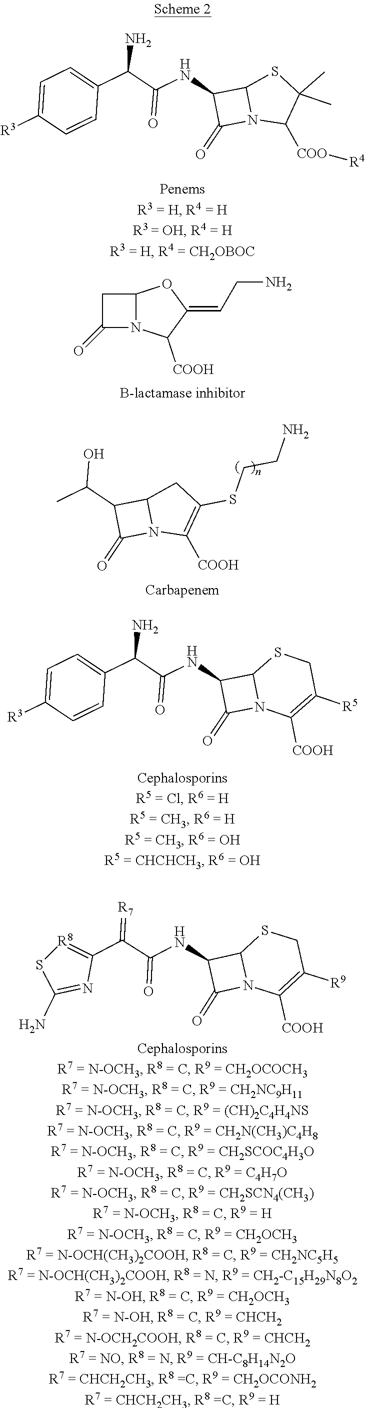

The linker A may be linked to the β-lactam ring of the cephalosporin, penem or carbapenem sub-structures or moieties via an amide linkage on the position a to the carbonyl of the β-lactam ring. In other words, the linker A may be linked to the β-lactam moiety L by an amide linkage on the α position to the carbonyl of the β-lactam ring.

Examples of the metallo-β-lactamase (MBL) inhibitors of the invention comprising a functionalised aza-cycloalkane zinc chelating moiety coupled via a linker to a cephalosporin sub-structure or moiety are shown in Scheme 3.

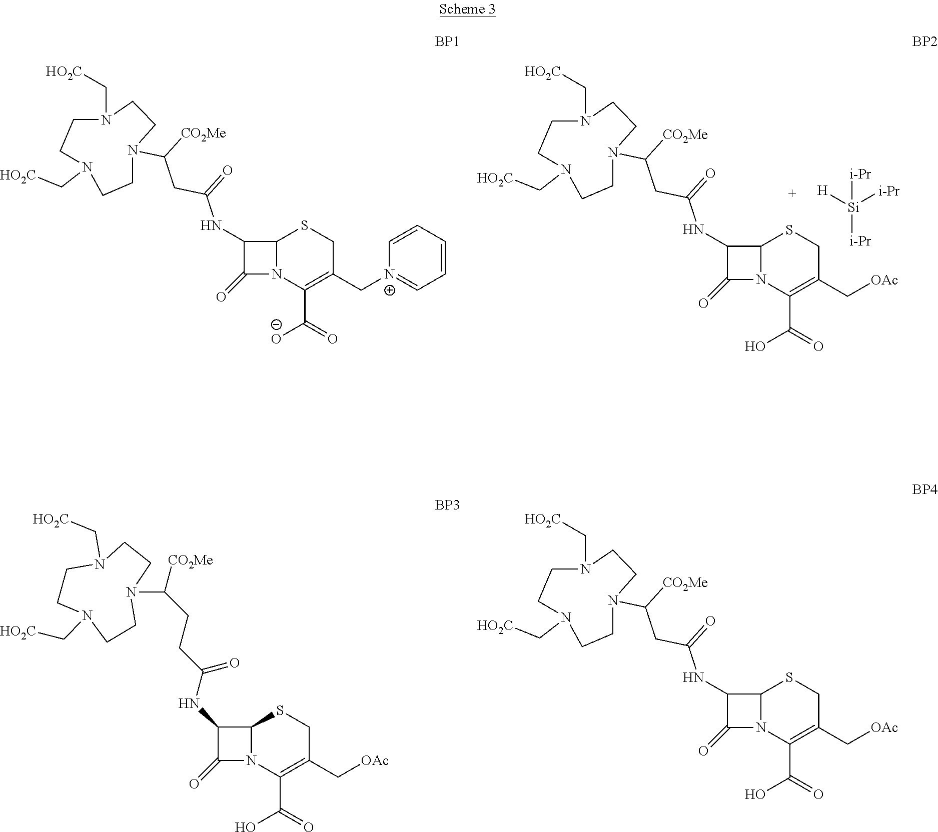

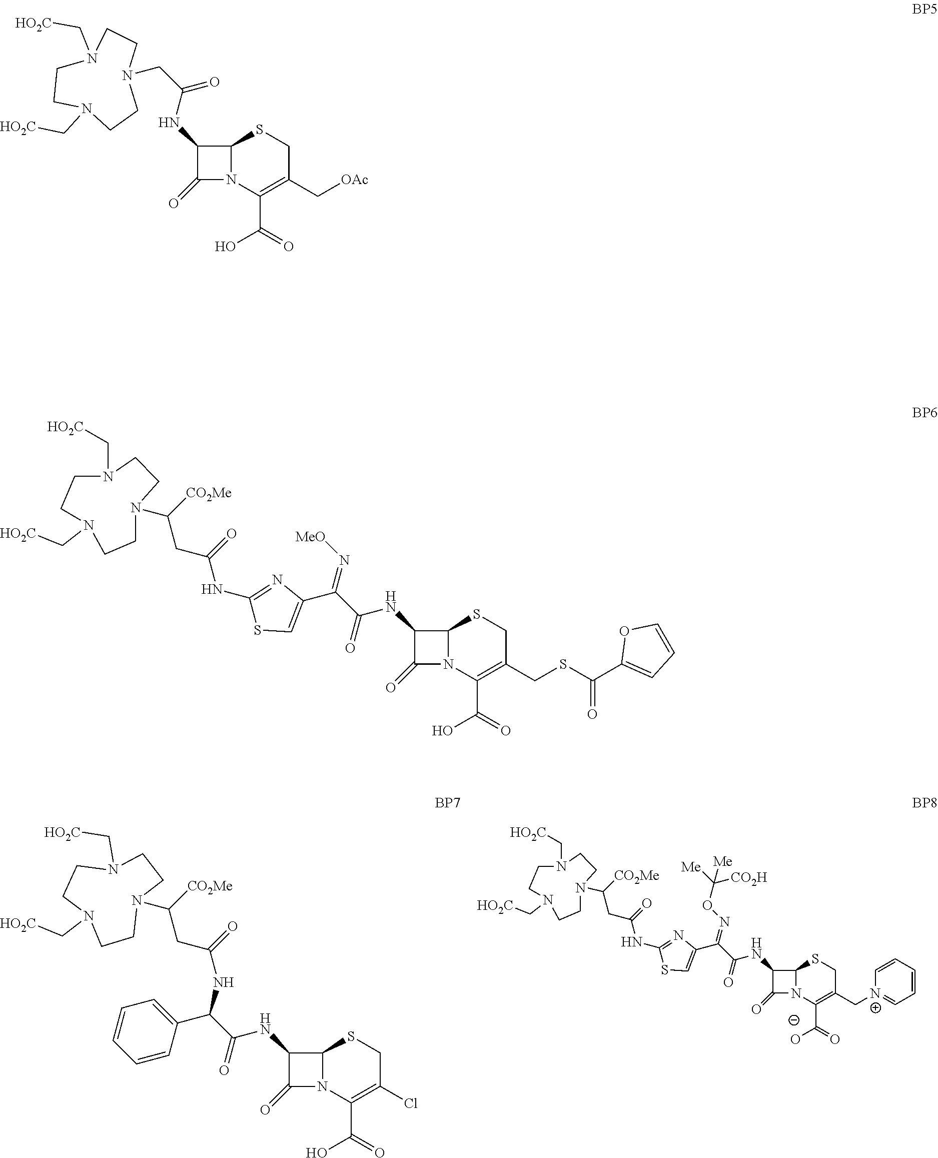 [SMILES: CC(=O)OCC1=C(C(=O)O)N2C(=O)[C@@H](NC(=O)CN3CCN(CC(=O)O)CCN(CC(=O)O)CC3)[C@H]2SC1 ; CO/N=C(/C(=O)N[C@@H]1C(=O)N2C(C(=O)O)=C(CSC(=O)c3ccco3)CS[C@H]12)c1csc(NC(=O)CC(C(=O)OC)N2CCN(CC(=O)O)CCN(CC(=O)O)CC2)n1 ; COC(=O)C(CC(=O)N[C@@H](C(=O)N[C@@H]1C(=O)N2C(C(=O)O)=C(Cl)CS[C@H]12)c1ccccc1)N1CCN(CC(=O)O)CCN(CC(=O)O)CC1 ; COC(=O)C(CC(=O)Nc1nc(/C(=N\OC(C)(C)C(=O)O)C(=O)N[C@@H]2C(=O)N3C(C(=O)[O-])=C(C[n+]4ccccc4)CS[C@H]23)cs1)N1CCN(CC(=O)O)CCN(CC(=O)O)CC1]

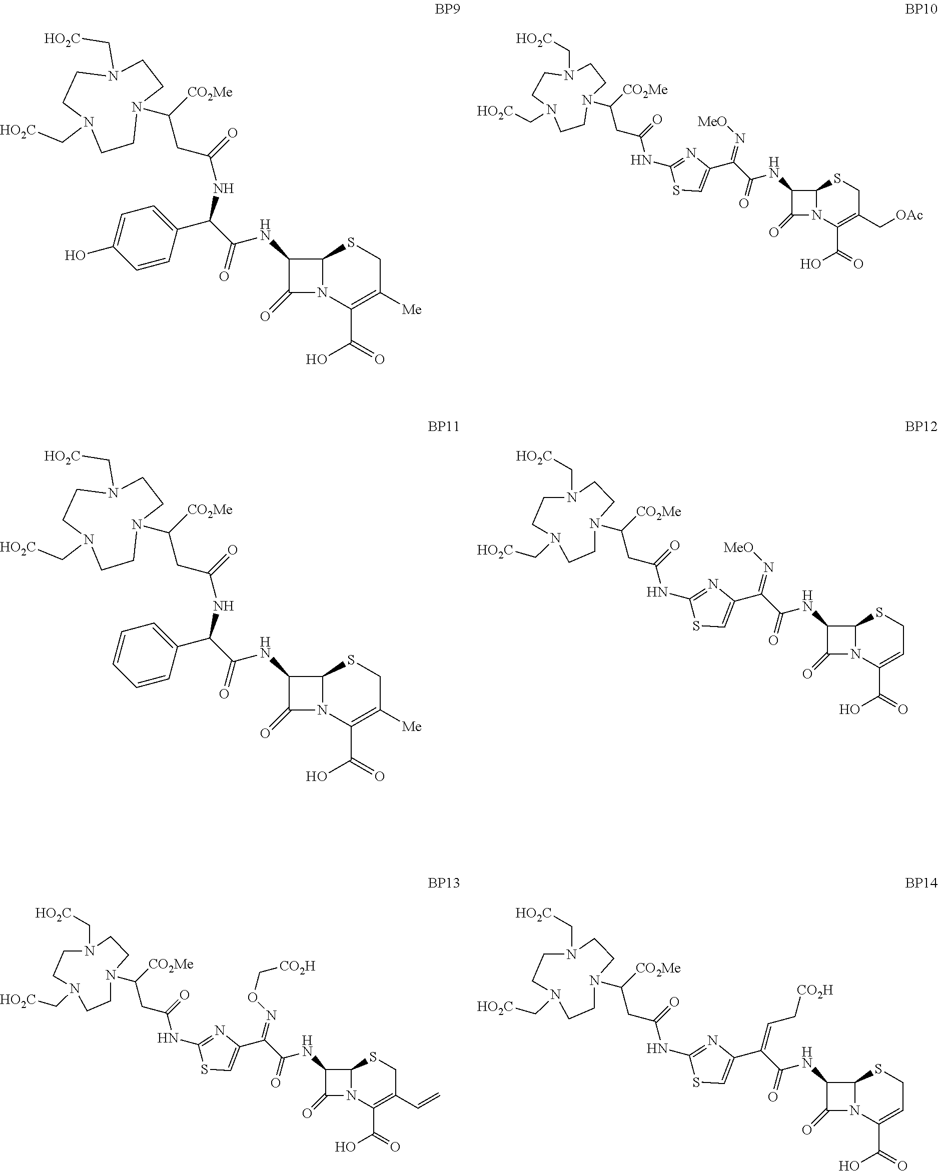

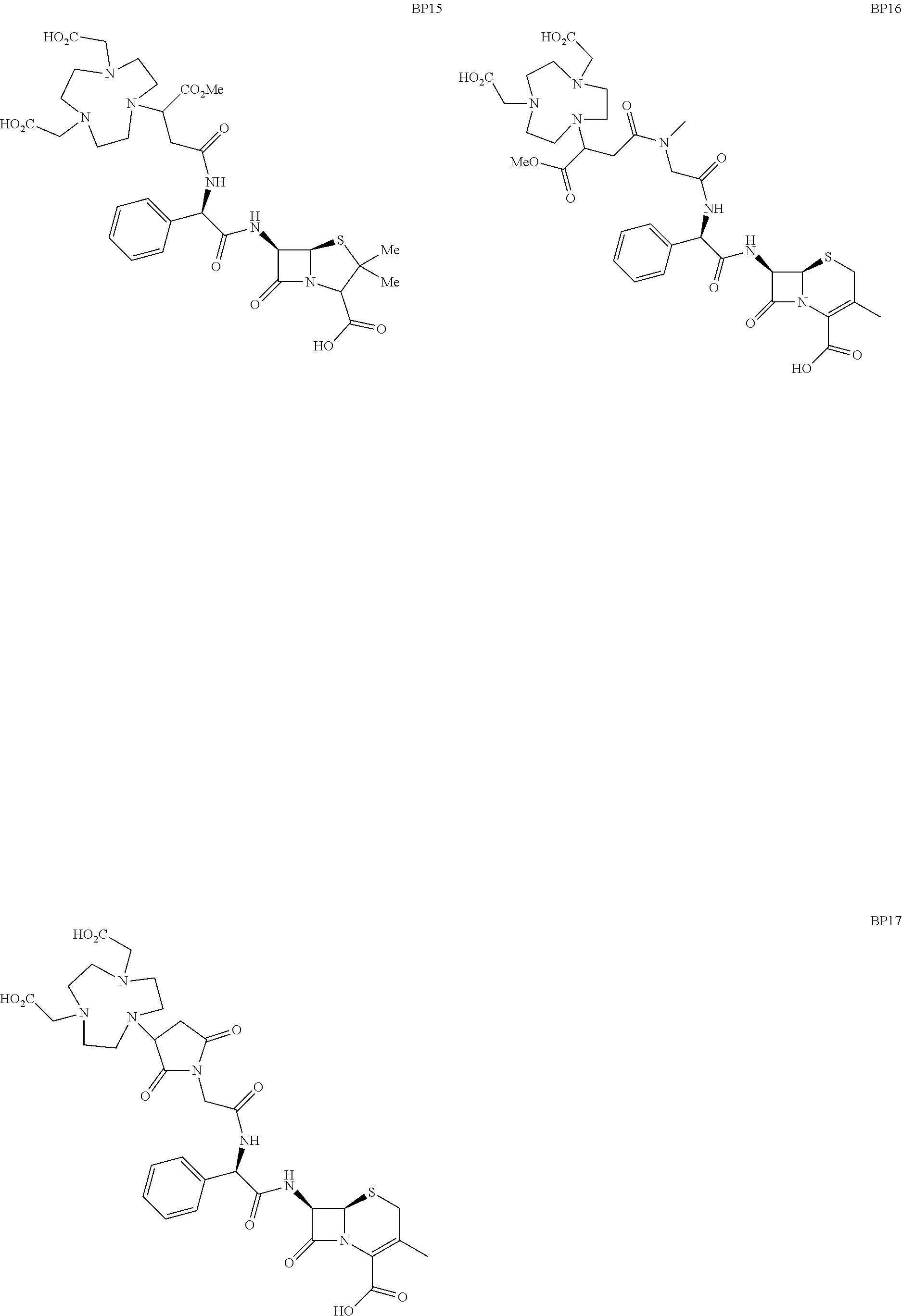

The invention extends to a pharmaceutical composition comprising a metallo-β-lactamase (MBL) inhibitor of general formula (I) and one or more pharmaceutically acceptable carriers or excipients.

ACCORDING TO A FURTHER ASPECT OF THE INVENTION there is provided the use of a metallo-β-lactamase (MBL) inhibitor of general formula (I) in the manufacture of a medicament for the treatment or prevention of a bacterial infection in a human or in a non-human mammal.

ACCORDING TO ANOTHER ASPECT OF THE INVENTION there is provided a metallo-β-lactamase (MBL) inhibitor of general formula (I), or a pharmaceutical composition as hereinbefore described, for use in a method of treating or preventing a bacterial infection in a human or in a non-human mammal.

ACCORDING TO YET A FURTHER ASPECT OF THE INVENTION there is provided a method of treating or preventing a bacterial infection in a human or in a non-human mammal, said method including the step of administering an effective amount of a metallo-β-lactamase (MBL) inhibitor of general formula (I) to said human or to said non-human mammal.

The method may include also administering one or more β-lactam antibiotics to said human or to said non-human mammal. Thus, the method may comprise the step of administering a metallo-β-lactamase (MBL) inhibitor of general formula (I) or the pharmaceutical composition comprising the metallo-β-lactamase (MBL) inhibitor of general formula (I) simultaneously, separately, or sequentially with a β-lactam antibiotic.

The β-lactam antibiotic may be selected from the group consisting of meropenem, imipenem, doripenem, ertapenem and combinations thereof.

ACCORDING TO YET ANOTHER ASPECT OF THE INVENTION there is provided a kit for the treatment or prevention of a bacterial infection in a human or non-human mammal, said kit comprising at least one of a metallo-β-lactamase (MBL) inhibitor of general formula (I), or a pharmaceutical composition as hereinbefore described.

The kit may further include one or more β-lactam antibiotics.

The one or more β-lactam antibiotics may be selected from the group consisting of meropenem, imipenem, doripenem, ertapenem and combinations thereof.

The bacterial infection may be an infection caused by bacterial strains such as E. coli IMP-1, E. coli NDM-1, E. coli VIM-1, E. cloacae VIM-1, E. cloacae IMP-1, K. pneumoniae IMP-8. K. pneumoniae VIM-1, K. pneumoniae IMP-1, K. pneumoniae NDM or P. rettgeri NDM and other strains containing or producing metallo beta lactamases.

The synthesis of compound BP1 (chelator to a cephalosporin, compound 4 of Scheme 4) is set out in Scheme 4.

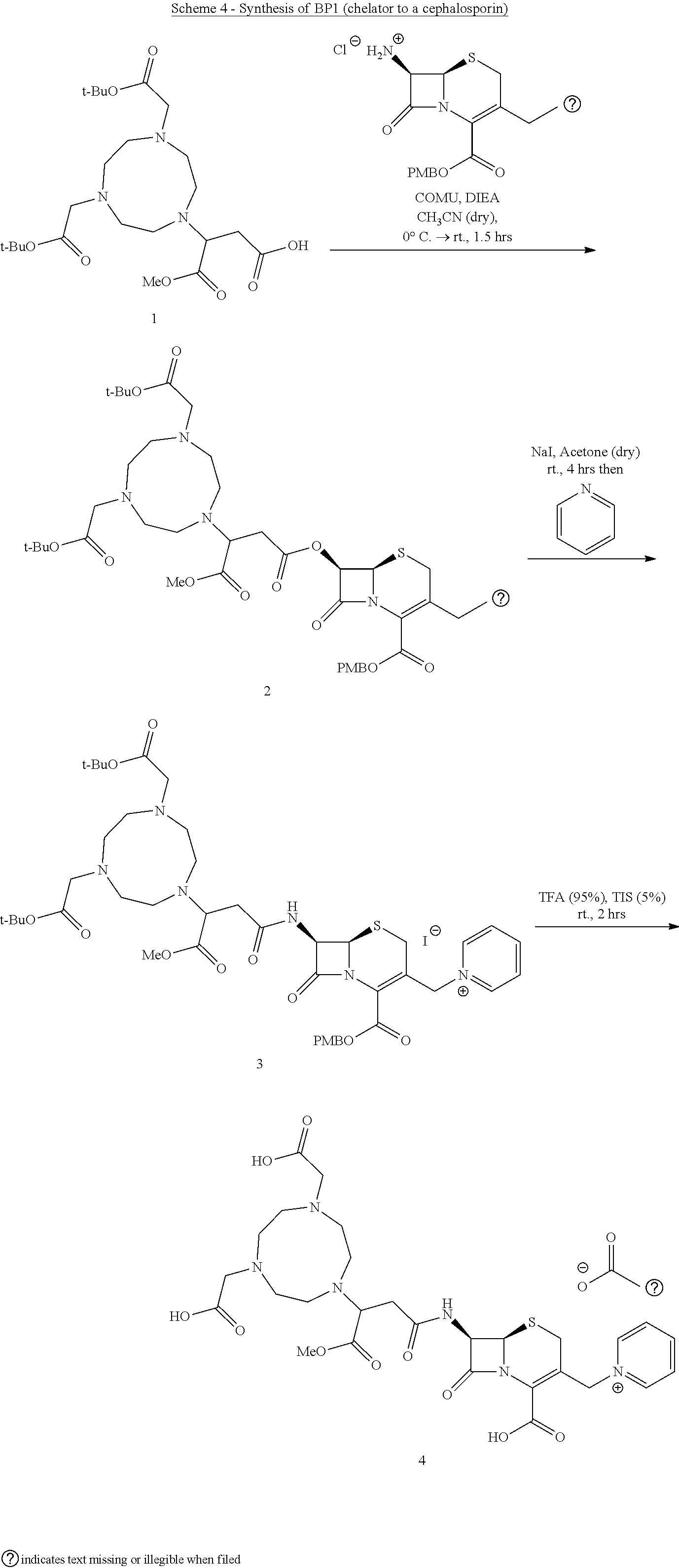

BP1 (compound 4 of Scheme 4) was synthesized in two steps from 1,4,7-triazacyclononane (1) using the peptide coupling agent COMU in the presence of diethylamine to produce the coupled product (3) and the protecting groups were subsequently removed using TFA to produce the de-protected final product (4).

Compound BP2 (chelator to a cephalosporin, compound 7 of Scheme 5) and compound BP3 (chelator to a penem, compound 10 of Scheme 6) were prepared similarly as shown in Schemes 5 and 6 below.

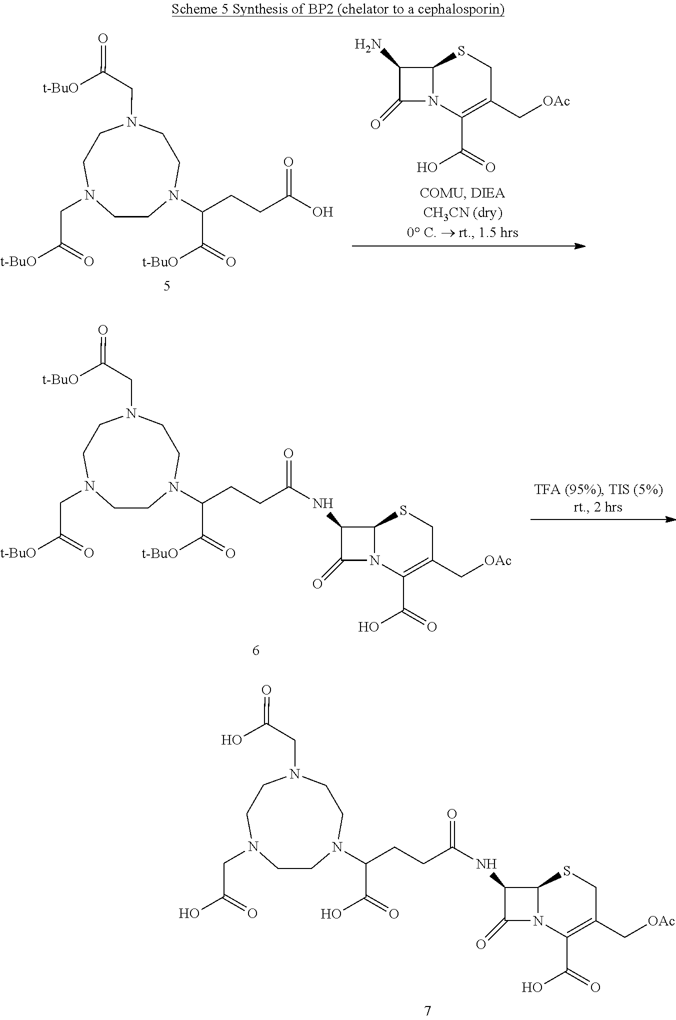 [SMILES: CC(C)(C)OC(=O)CN1CCN(CC(=O)OC(C)(C)C)CCN(C(CCC(=O)O)C(=O)OC(C)(C)C)CC1 ; CC(=O)OCC1=C(C(=O)O)N2C(=O)[C@@H](N)[C@H]2SC1 ; CC(=O)OCC1=C(C(=O)O)N2C(=O)[C@@H](NC(=O)CCC(C(=O)OC(C)(C)C)N3CCN(CC(=O)OC(C)(C)C)CCN(CC(=O)OC(C)(C)C)CC3)[C@H]2SC1 ; CC(=O)OCC1=C(C(=O)O)N2C(=O)[C@@H](NC(=O)CCC(C(=O)O)N3CCN(CC(=O)O)CCN(CC(=O)O)CC3)[C@H]2SC1]

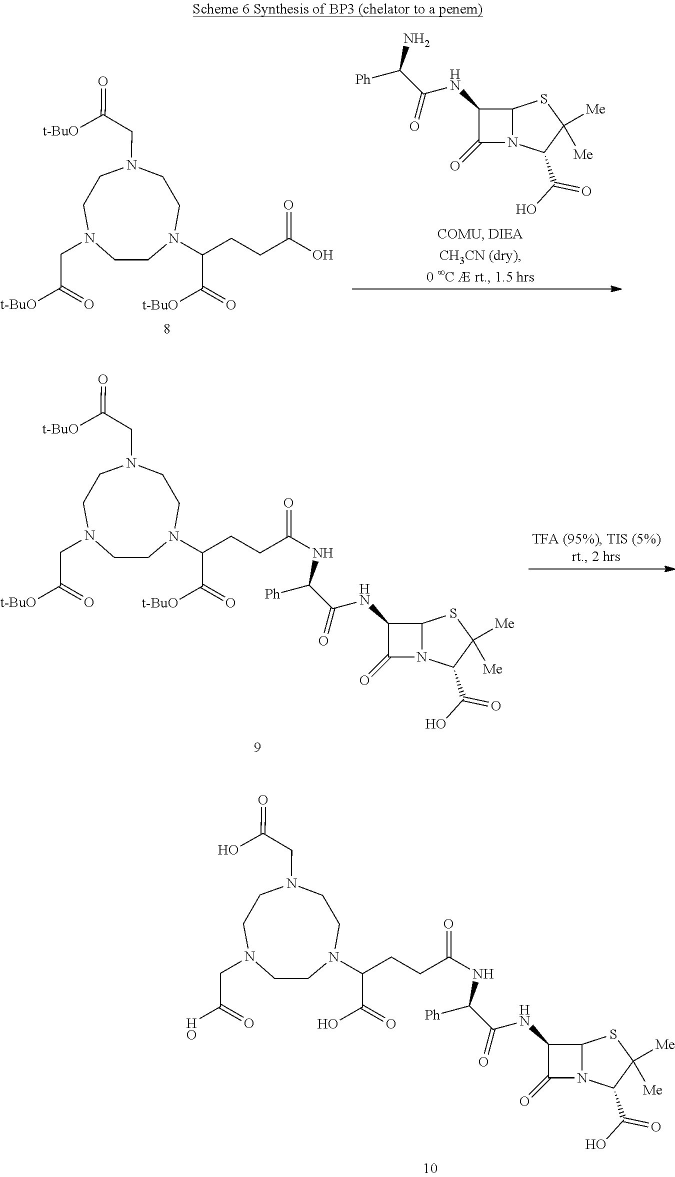 [SMILES: CC(C)(C)OC(=O)CN1CCN(CC(=O)OC(C)(C)C)CCN(C(CCC(=O)O)C(=O)OC(C)(C)C)CC1 ; CC1(C)SC2[C@H](NC(=O)[C@H](N)c3ccccc3)C(=O)N2[C@H]1C(=O)O ; CC(C)(C)OC(=O)CN1CCN(CC(=O)OC(C)(C)C)CCN(C(CCC(=O)N[C@@H](C(=O)N[C@@H]2C(=O)N3C2SC(C)(C)[C@@H]3C(=O)O)c2ccccc2)C(=O)OC(C)(C)C)CC1 ; CC1(C)SC2[C@H](NC(=O)[C@H](NC(=O)CCC(C(=O)O)N3CCN(CC(=O)O)CCN(CC(=O)O)CC3)c3ccccc3)C(=O)N2[C@H]1C(=O)O]

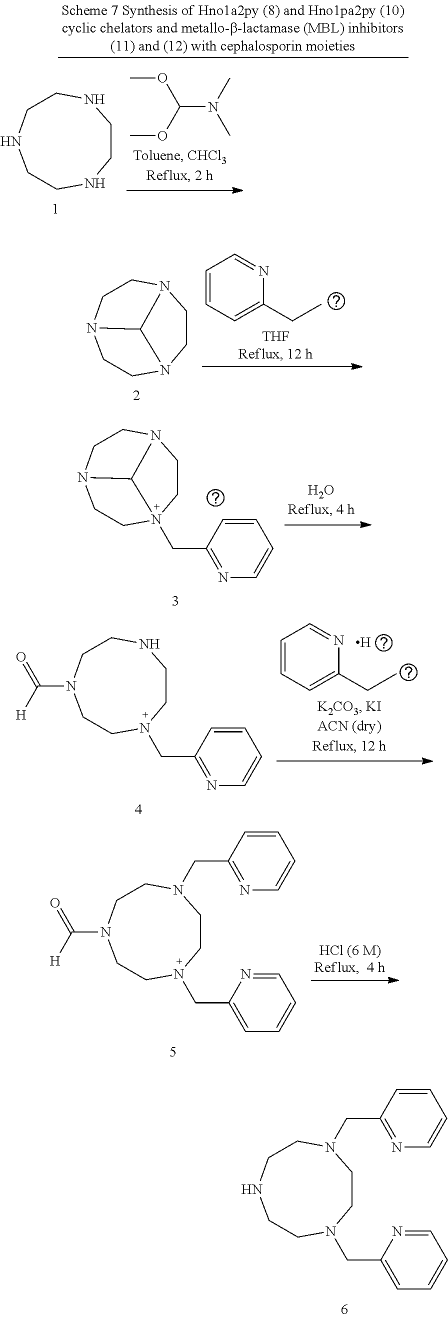

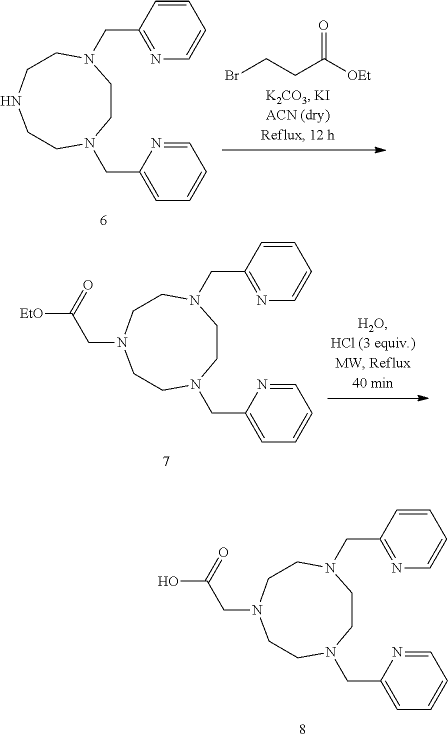 [SMILES: c1ccc(CN2CCNCCN(Cc3ccccn3)CC2)nc1 ; CCOC(=O)CCBr ; CCOC(=O)CN1CCN(Cc2ccccn2)CCN(Cc2ccccn2)CC1 ; O=C(O)CN1CCN(Cc2ccccn2)CCN(Cc2ccccn2)CC1]

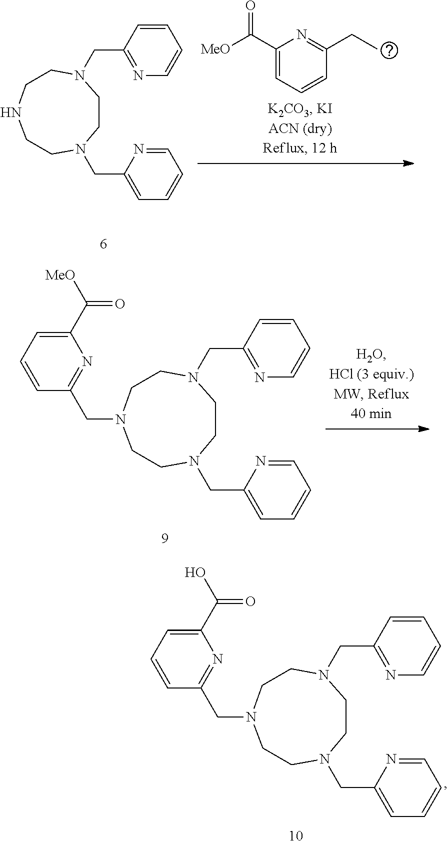

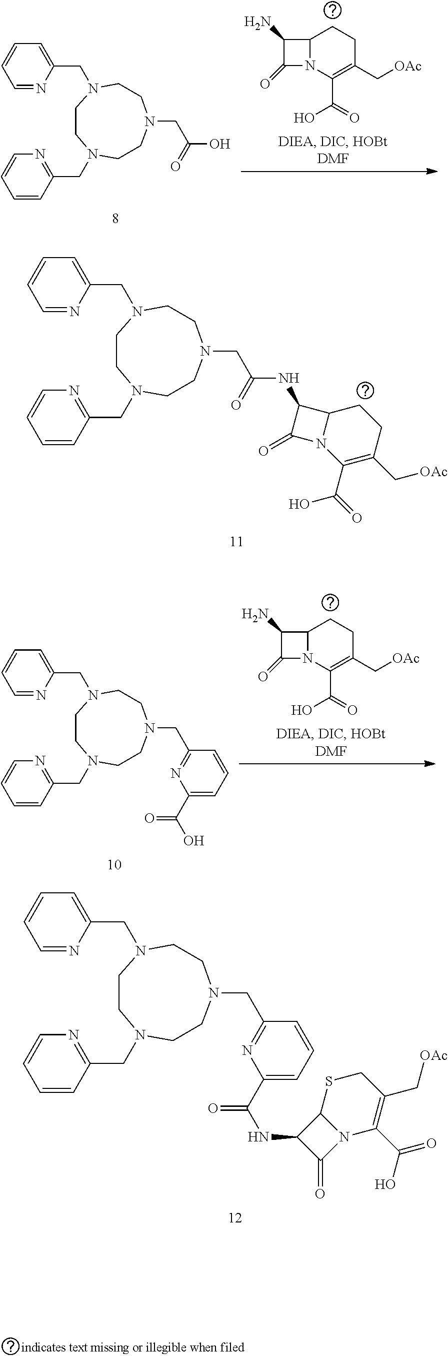 [SMILES: O=C(O)CN1CCN(Cc2ccccn2)CCN(Cc2ccccn2)CC1 ; CC(=O)OCC1=C(C(=O)O)N2C(=O)[C@@H](N)C2CC1 ; CC(=O)OCC1=C(C(=O)O)N2C(=O)[C@@H](NC(=O)CN3CCN(Cc4ccccn4)CCN(Cc4ccccn4)CC3)C2CC1 ; O=C(O)c1cccc(CN2CCN(Cc3ccccn3)CCN(Cc3ccccn3)CC2)n1 ; CC(=O)OCC1=C(C(=O)O)N2C(=O)[C@@H](N)C2CC1 ; CC(=O)OCC1=C(C(=O)O)N2C(=O)[C@@H](NC(=O)c3cccc(CN4CCN(Cc5ccccn5)CCN(Cc5ccccn5)CC4)n3)C2SC1]

Other BP compounds disclosed herein but the synthesis of which is not specifically described were prepared similarly to the BP compounds the synthesis of which is specifically described herein.

### DETAILED DESCRIPTION OF THE INVENTION

THE INVENTION IS NOW DESCRIBED BY WAY OF EXAMPLE with reference to the following Examples, Figures and Tables in which:

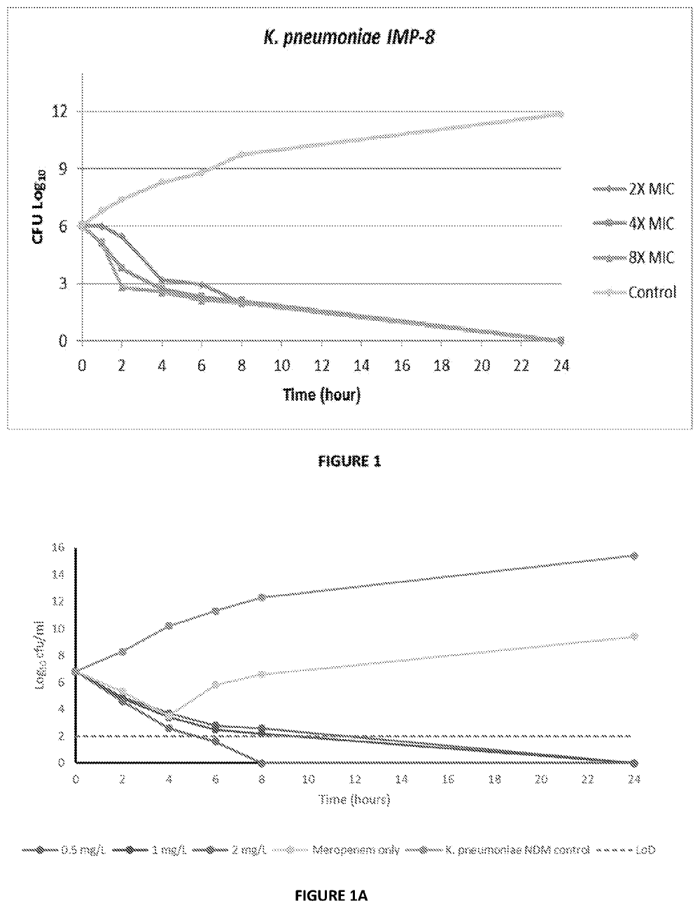

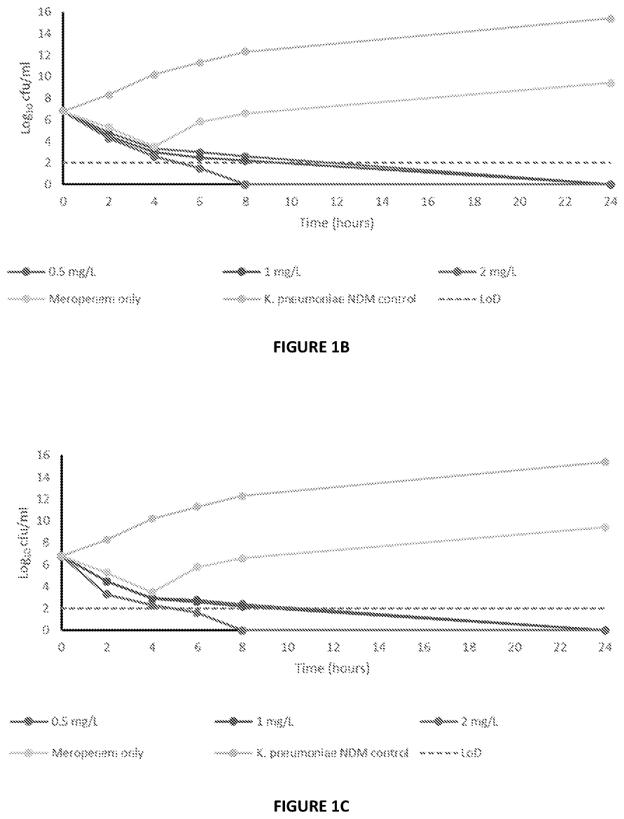

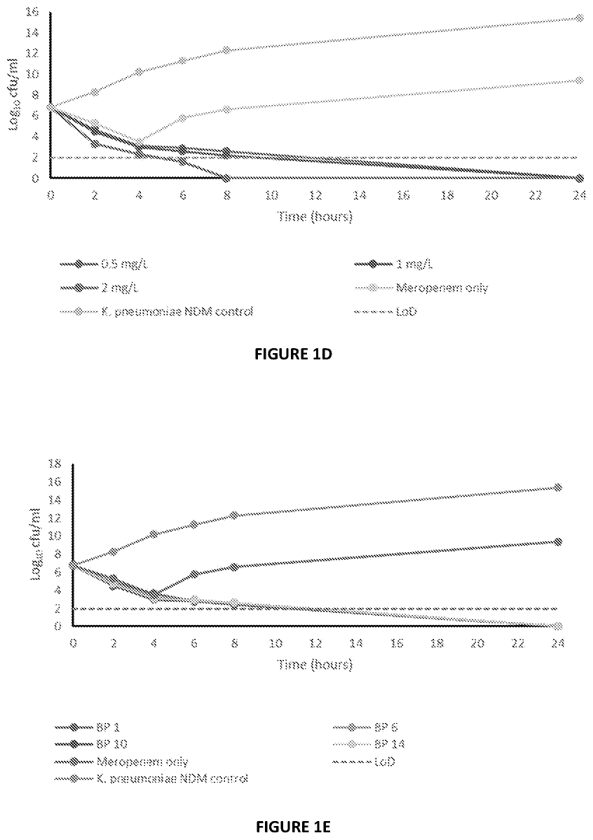

FIG. 1 shows a trace of time kill kinetics as a function of time at different concentrations of meropenem and BP1 for K. pneumoniae IMP-8;

FIG. 1A shows a trace of time kill kinetics as a function of time at different concentrations of meropenem and BP1 for K. pneumoniae NDM;

FIG. 1B shows a trace of time kill kinetics as a function of time at different concentrations of meropenem and BP6 for K. pneumoniae NDM;

FIG. 1C shows a trace of time kill kinetics as a function of time at different concentrations of meropenem and BP10 for K. pneumoniae NDM;

FIG. 1D shows a trace of time kill kinetics as a function of time at different concentrations of meropenem and BP14 for K. pneumoniae NDM;

FIG. 1E shows a trace of time kill kinetics as a function of time at different concentrations of meropenem and BP1, BP6, BP10 and BP14 for K. pneumoniae NDM. This graph shows a fixed concentration of each BP compound (32 mg/L) with the lowest concentration of meropenem (0.5 mg/L) studied;

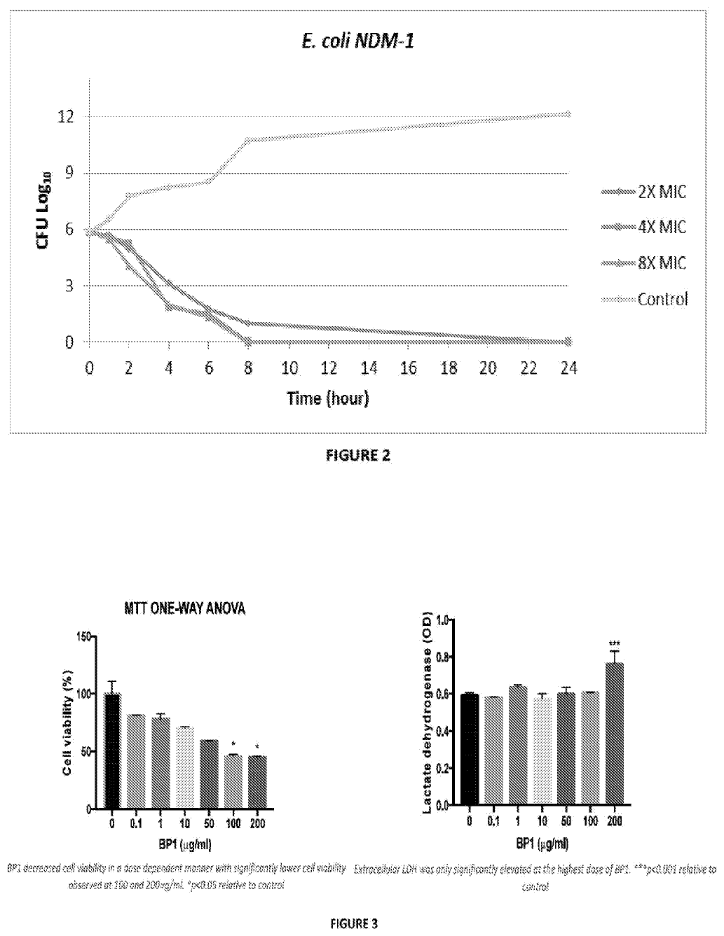

FIG. 2 shows a trace of time kill kinetics as a function of time at different concentrations of meropenem and BP1 for E. coli NDM-1;

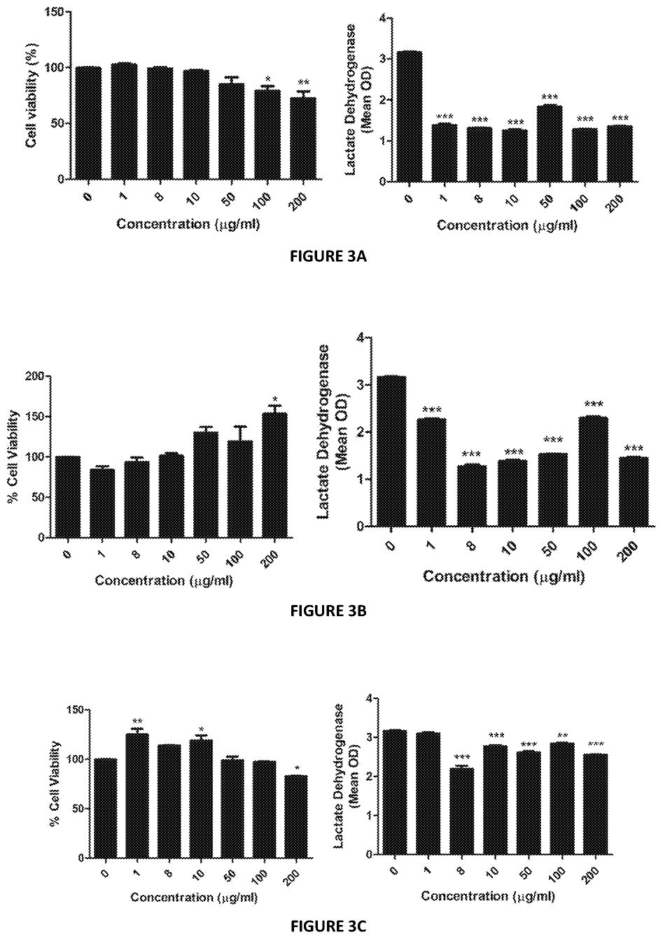

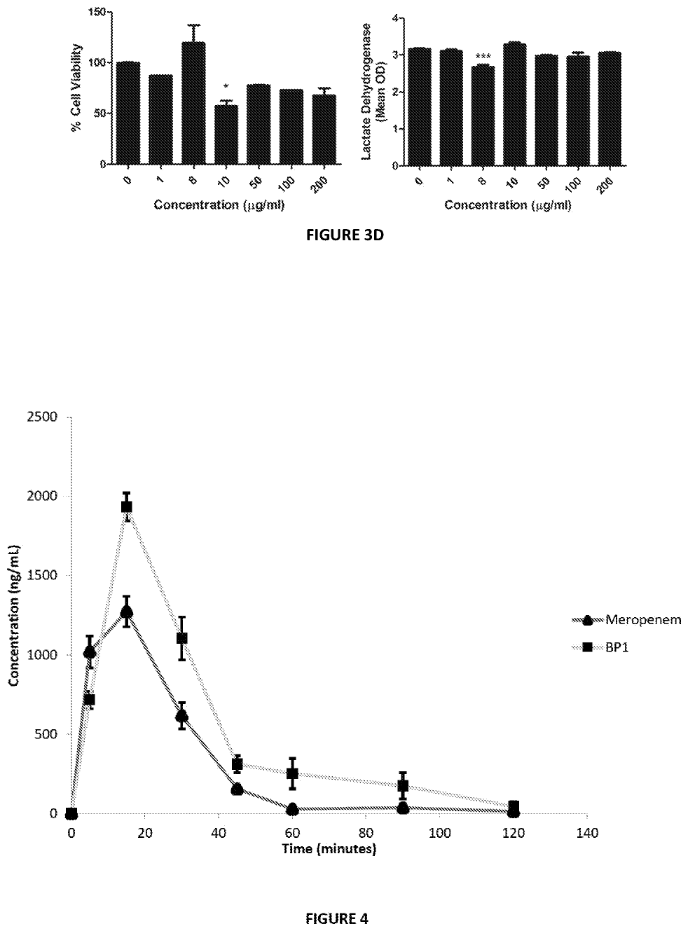

FIG. 3 shows two bar graphs of HepG2 cell viability as a function of dosage of BP1 and lactate dehydrogenase as a function of dosage of BP1;

FIG. 3A shows two further bar graphs of HepG2 cell viability as a function of dosage of BP1 and lactate dehydrogenase as a function of dosage of BP1. The nonlinear fit, of BP1 on HepG2 cells generated an IC₅₀ value of 59.96 μg/ml. A dose-dependent decrease in cell viability was observed with a significant decrease occurring at 100 and 200 μg/ml. *p<0.5 and **p<0.01 relative to control. Extracellular lactate dehydrogenase (LDH), was significantly reduced at all concentrations, indicating that BP1 does not induce necrosis after exposure. ***p<0.0001 relative to control;

FIG. 3B shows two bar graphs of HepG2 cell viability as a function of dosage of BP6 and lactate dehydrogenase as a function of dosage of BP6. BP6 induced a dose dependent increase in the cell viability of HepG2 cells, however cell viability was only significantly altered at 200 μg/ml. *p<0.05 relative to control. LDH, was significantly reduced at all concentrations, indicating that BP6 does not induce necrosis after exposure. ***p<0.0001 relative to control;

FIG. 3C shows two bar graphs of HepG2 cell viability as a function of dosage of BP10 and lactate dehydrogenase as a function of dosage of BP10. Cell viability was not significantly altered at 50-100 μg/ml, however significantly reduced at 200 μg/ml. *p<0.5 and **p<0.01 relative to control. LDH levels remained unaffected at 1 μg/ml and were significantly reduced at 8-200 μg/ml, indicating BP10 does not induce necrosis in HepG2 cells after exposure. **p<0.01 and **p<0.001 relative to control;

FIG. 3D shows two bar graphs of HepG2 cell viability as a function of dosage of BP14 and lactate dehydrogenase as a function of dosage of BP14. BP-14 reduced cell viability at 1 μg/ml and 10-200 μg/ml; however, proliferation occurred at 8 μg/ml. *p<0.5 and **p<0.01 relative to control. LDH levels were significantly reduced at 8 μg/ml and remained unaffected at 1 μg/ml and 10-200 μg/ml, indicating BP-14 does not induce necrosis in HepG2 cells after exposure. **p<0.001 relative to control;

FIG. 4 shows pharmacokinetics of BP1 and meropenem in a healthy murine model, data is presented as a mean±SD (n=3);

FIG. 5 shows changes in thigh CFU counts following treatment with BP1 over a 24-hour period, data is presented as a mean±SD (n=3); and

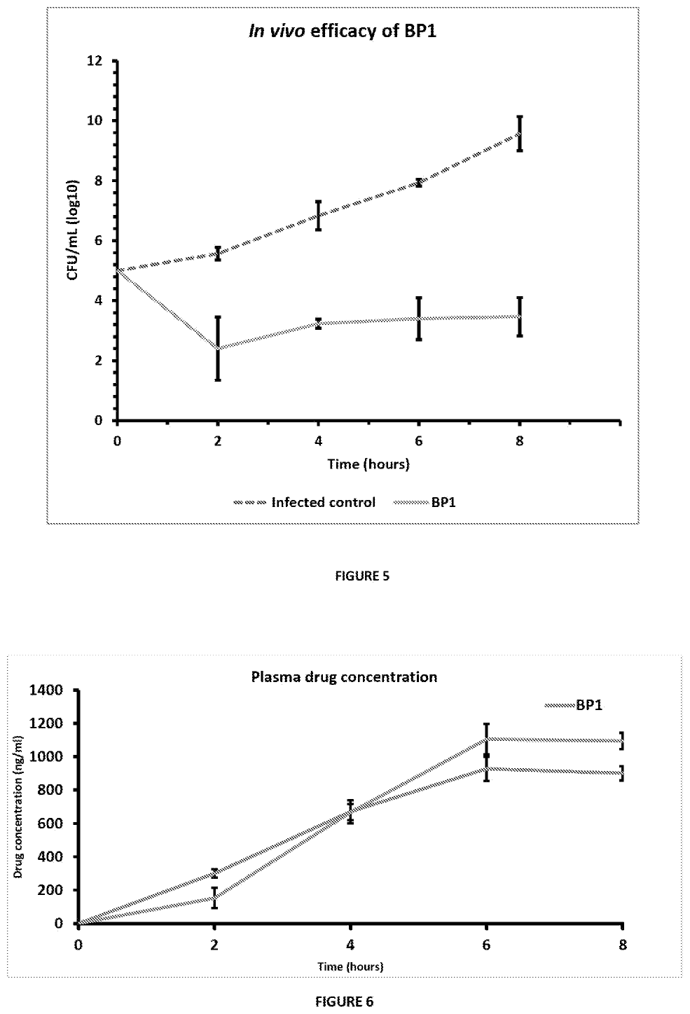

FIG. 6 shows plasma BP1 concentrations over an 8-hour treatment period, data is presented as a mean±SD (n=3).

### DISCUSSION

In the discussion below “BP1” refers to compound 4 of Scheme 4 and “TG1” refers to 1,4,7-triazacyclononane-1,4,7-triacetic acid (NOTA).

### Materials

Meropenem and human serum were purchased from Sigma-Aldrich.

### Bacterial Strains

Reference strains of MBL-producing CRE were bought from Nordmann (Nordmann P. Rapid Detection of Carbapenemase-producing Enterobacteriaceae—Volume 18, Number 9—September 2012-Emerging Infectious Disease journal-CDC. 2012) at the Institut National de la Santé et de la Recherche Médicale, Paris, France. The reference strain used as quality control was Escherichia coli ATCC 25922.

### Synthesis of Compounds 4, 7 and 10

### General Information

All the chemicals were purchased from commercial sources and used without further purification. Mono-methyl fumarate, N,N-diisopropylethylamine (DIEA), tetrahydrofuran (THF) and dichloromethane (CH₂Cl₂) were purchased form Sigma-Aldrich.

All compounds were analyzed by RP-HPLC (Agilent 1100, USA). Runs were performed using a linear gradient of solvent A (0.1% TFA in H₂O) and solvent B (0.1% TFA in ACN); where the gradient was 5% B to 95% B in 15 min at a flow rate of 1 ml/min. All the synthetic steps were further characterized using LC/MS (Shimadzu 2020 UFLC-MS, Japan) with an YMC-Triart C18 (5 μm, 4.6×150 mm) column for their respective masses. NMR spectra (¹H NMR, ¹³C NMR, HMBC and HSQC) were recorded on a Bruker AVANCE III 400 MHz spectrometer using deuterated methanol as the solvent. HRMS was carried out on a Bruker microTOF-QII.

### Preparation of Compounds 2 (Scheme 4), 6 (Scheme 5) and 9 (Scheme 6)

Compounds 1 (Scheme 4), 5 (Scheme 5) or 8 (Scheme 6) (1.0 equiv.) were dissolved in dry ACN:THF (30 mL/mmol), N,N-diisopropylethylamine (3.0 equiv.) was then added in one shot, followed by DIC (2.0 equiv.). After 5 minutes 7-Amino-3-chloromethyl-3-cephem-4-carboxylic acid p-methoxybenzyl ester hydrochloride (Scheme 1, 1→2), 7-Aminocephalosporanic acid (Scheme 5, 5→6) or Ampicillin (Scheme 6, 8→9) (1.0 equiv.) was added and the progress of the reaction was then monitored by LC/MS. After 24 hours, the reaction was complete. Purification of compound 2 of Scheme 4 was performed by elution through a plug of alumina with EtOAc:Hexane (1:1), 83% yield. Compounds 6 (Scheme 5) and 9 (Scheme 6) were purified using prep-SFC with the following parameters: sample concentration=8 mg/mL (ACN, 0.3% Et₃N), injection volume=100 μL, column=Ethylpyridine (250×10 mm, 5 Å) at 40° C., mobile phase=10-50% MeOH:ACN:Et₃N (99.85:99.85:0.3) as the modifier, tech grade-wet CO₂ the balance of the flow, in 10 min, flow=10 mL/min, stacked injection program with a two minute equilibration time, BPR setting=150 bar, monitoring and collection at 220 nm. Yields were 50-70%.

### Preparation of Compound 3 of Scheme 4

The procedure was adapted from that of Long et al (D. D. Long, J. B. Aggen, J. Chinn, S.-K. Choi, B. G. Christensen, P. R. Fatheree, D. Green, S. S. Hegde, J. K. Judice, K. Kaniga, K. M. Krause, M. Leadbetter, M. S. Linsell, D. G. Marquess, E. J. Moran, M. B. Nodwell, J. L. Pace, S. G. Trapp and S. D. Turner, J. Antibiot., 2008, 61, 603-614). Compound 2 of Scheme 4 (1.0 equiv.) was dissolved in dry acetone (4 mL/mmol to compound 2 of Scheme 4) and the flask was covered with aluminum foil to shield the reaction from light. Sodium iodide (1.0 equiv.) was then added, in one shot at room temperature and the reaction stirred for 1.5 hours, before pyridine (1.1 equiv.) was added. The reaction was stirred for another 1.5 hours (progress of this step monitored by LC/MS). Quick inspection revealed that the solution had turned to a red/purple. The solvent was then removed, producing a red/purple foam. The foam was sonicated (5 minutes) in Et₂O (5 mL/mmol to compound 2 of Scheme 4) and the washing decanted, this was repeated two times. The purity of the wash >90%, however, further purification was carried out using prep-SFC with the following parameters: sample concentration=16 mg/mL (ACN, 0.3% Et₃N), injection volume=100 μL, column=Ethylpyridine (250×10 mm, 5 Å) at 40° C., mobile phase=10-50% MeOH:ACN:Et₃N (99.85:99.85:0.3) as the modifier, tech grade-wet CO₂ the balance of the flow, in 10 min, flow=10 mL/min, stacked injection program with a two minute equilibration time, BPR setting=150 bar, monitoring and collection at 220 nm. Compound 7 of Scheme 5 was obtained in 45% yield as a light yellow solid, based on the compound 3. Et₃N salt.

### Preparation of Compounds 4, 7 and 10

To compound 3 (Scheme 4), compound 6 (Scheme 5) or compound 9 (Scheme 6) (1.0 equiv.) was added triisopropylsilane (4 mL/mmol to 7) and then TFA (76 mL/mmol to compounds 3, 6 or 9). The reaction was allowed under stirring for 20 min at room temperature, after which the volatiles were removed by passing a gentle stream of nitrogen over the reaction until a pale-yellow residue remained. The residue was sonicated (10 minutes) in Et₂O (5 mL/mmol to compounds 3, 6 or 9) and the washing decanted, giving a fine precipitate. This was repeated two more times with Et₂O, producing pure compound 4 (Scheme 4), compound 7 (Scheme 5) and compound 10 (Scheme 6) in 85-95% yield as white solids.

### Synthesis of Compounds 2, 3, 4, 5 and 6 of Scheme 7

### General Information

All the chemicals were purchased from commercial sources and used without further purification. Mono-methyl fumarate, N,N-Diisopropylethylamine (DIEA), Tetrahydrofuran (THF) and Dichloromethane (CH₂Cl₂) were purchased form Sigma-Aldrich.

All compounds were analyzed by RP-HPLC (Agilent 1100, USA). All the synthetic steps were further characterized using LC/MS (Shimadzu 2020 UFLC-MS, Japan) with an YMC-Triart C18 (5 μm, 4.6×150 mm) column for their respective masses. NMR spectra (¹H NMR, ¹³C NMR, HMBC and HSQC) were recorded on a Bruker AVANCE III 400 MHz spectrometer using deuterated chloroform or methanol as the solvent. HRMS was carried out on a Bruker microTOF-QII.

Compound 2 of Scheme 7 was prepared using a procedure adapted from Roger, M., et al., Monopicolinate-dipicolyl Derivative of Triazacyclononane for Stable Complexation of Cu2+ and 64Cu2+. Inorganic Chemistry, 2013. 52(9): p. 5246-5259.

(1→2). 1,4,7-triazacyclononane (compound 1 of Scheme 7) (1.0 equiv.) was dissolved in a mixture of toluene and chloroform (1.3 and 0.3 ml/mmol respectively). Dimethoxymethyl-N,N-dimethylamine (1.0 equiv.) was added, in a single shot and the mixture was refluxed. NMR was used to monitor the progress of the reaction and it took 2 hours to complete. The solvent was then removed producing pure compound 2 of Scheme 7 in 85-95% yield as a yellow oil.

Compound 3 of Scheme 7 was prepared using a procedure adapted from Gasser, G., et al., Synthesis, Copper(II) Complexation, 64Cu-Labeling, and Bioconjugation of a New Bis(2-pyridylmethyl) Derivative of 1,4,7-Triazacyclononane. Bioconjugate Chem., 2008. 19(3): p. 719-730.

(2→3). To compound 2 of Scheme 7 (1.0 equiv.) dissolved in dryTHF (1 ml/mmol) a solution of picolyl chloride (1.0 equiv.) in THF was added drop-wise cautiously. The reaction was stirred overnight at room temperature to afford a red-orange precipitate. The liquid was decanted and the precipitate was sonicated (5 minutes) in THF (5 mL/mmol to compound 2 of Scheme 7) and the washing decanted. This was repeated two times. The precipitate was dried to yield compound 3 of Scheme 7 in 75-86% yield.

Compound 4 of Scheme 7 was prepared using a procedure adapted from Gasser, G., et al., Synthesis, Copper(II) Complexation, 64Cu-Labeling, and Bioconjugation of a New Bis(2-pyridylmethyl) Derivative of 1,4,7-Triazacyclononane. Bioconjugate Chem., 2008. 19(3): p. 719-730.

Compound 3 was dissolved in milli Q Water (10 ml/mmol) and refluxed. The reaction was monitored by LC/MS. After 4 hours the reaction was complete. The pH of the mixture was adjusted to about 12 and extracted 3 times with CHCl₃. The organic extract was dried with MgSO₄ and concreted to afford compound 4 of Scheme 7 in 45-60% yield.

(4→5). Compound 5 of Scheme 7 was prepared using a procedure adapted from Gasser, G., et al., Synthesis, Copper(II) Complexation, 64Cu-Labeling, and Bioconjugation of a New Bis(2-pyridylmethyl) Derivative of 1,4,7-Triazacyclononane. Bioconjugate Chem., 2008. 19(3): p. 719-730.

Compound 4 of Scheme 7 was dissolved in dry ACN (1.4 mL/mmol), K₂CO₃ (4.0 equiv.) and KI (16.0 equiv.). The mixture was stirred at room temperature. Thereafter picolyl chloride hydrochloride solution in ACN (1.4 ml/mmol) was added drop-wise for 10-15 minutes. The reaction was further stirred for an hour at room temperature, followed by a reflux overnight. Thereafter the mixture was cooled and filtered. The aliquot was concentrated to afford compound 5 of Scheme 7 in 50-70% yields.

Compound 6 of Scheme 7 was prepared using a procedure adapted from Gasser, G., et al., Synthesis, Copper(II) Complexation, 64Cu-Labeling, and Bioconjugation of a New Bis(2-pyridylmethyl) Derivative of 1,4,7-Triazacyclononane. Bioconjugate Chem., 2008. 19(3): p. 719-730.

Compound 5 was dissolved in HCl 6M (10 ml/mmol) and refluxed. The reaction was monitored by LC/MS. After 4 hours the reaction was complete. Compound 6 of Scheme 7 was isolated similar to compound 4 of Scheme 7 with a 55-65% yield.

### Preparation of Compounds 8 (Hnola2py) and 10 (Hno1pa2py) of Scheme 7

A modified procedure was adapted from Gasser, G., et al., Synthesis, Copper(II) Complexation, 64Cu-Labeling, and Bioconjugation of a New Bis(2-pyridylmethyl) Derivative of 1,4,7-Triazacyclononane. Bioconjugate Chem., 2008. 19(3): p. 719-730.

Compound 6 of Scheme 7 (1.0 equiv.) was dissolved in dry ACN (1 mL/mmol), K₂CO₃ (0.4 equiv.) and KI (0.2 equiv.). The mixture was stirred at room temperature. Thereafter picolyl chloride hydrochloride (Scheme 7, 6→7) or 6-chloromethylpyridine-2-carboxylic acid methylester (Scheme 7, 6→9) solution in ACN (1 ml/mmol) was added drop-wise for 10-15 minutes. The reaction was further stirred for an hour at room temperature, followed by a reflux overnight. Thereafter the mixture was cooled and filtered. The aliquot was concentrated to afford compound 7 of Scheme 7 or compound 9 of Scheme 7 in yields of 50-70%. Compound 7 of Scheme 7 (7→8) (1 equiv.) or compound 9 of Scheme 7 (9→10) (1 equiv.) was diluted with Milli Q water 20-30 ml/mmol and HCl 6M (3 equiv.) was added. The reaction mixture was set to reflux at 100° C. in a microwave and the progress of the reaction was monitored by LC/MS. After 40 minutes, the reaction was complete. The mixture was further diluted five folds with water, frozen and the water removed with a freeze dry process to afford quantitative yields. 6-chloromethylpyridine-2-carboxylic acid methylester was prepared using a procedure adopted from Mato-Iglesias, M., et al., Lanthanide Complexes Based on a 1,7-Diaza-12-crown-4 Platform Containing Picolinate Pendants: A New Structural Entry for the Design of Magnetic Resonance Imaging Contrast Agents. Inorganic Chemistry, 2008. 47(17): p. 7840-7851.

### Preparation of Compounds 11 and 12 (Scheme 7)

Compounds 8 or 10 of Scheme 7 (1.0 equiv.) were dissolved in dry DMF (1-2 mL/mmol). N,N-diisoproplyethylamine (5.0 equiv.) was then added in one shot, followed by HOBt (1.2 equiv.) and DIC (1.2 equiv.). After 5 minutes, 7-Aminocephalosporanic acid (5→6) was added and the progress of the reaction was then monitored by LC/MS. After overnight, the reaction was complete. Compounds 11 and 12 of Scheme 7 were purified using prep-SFC with the following parameters: sample concentration=10 mg/mL (ACN, 0.3% Et₃N), injection volume=100 μL, column=Ethylpyridine (250×10 mm, 5 Å) at 40° C., mobile phase=10-50% MeOH:ACN:Et₃N (49.85:49.85:0.3) as the modifier, tech grade-wet CO₂ the balance of the flow, in 10 min, flow=10 mL/min, stacked injection program with a two minute equilibration time, BPR setting=150 bar, monitoring and collection at 220 nm.

### Minimum Inhibitory Concentrations (MICs)

Minimum inhibitory concentration (MIC) determination was performed according to the CLSI guidelines and Keepers et al (Keepers T R, Gomez M, Celeri C et al. Bactericidal activity, absence of serum effect, and time-kill kinetics of ceftazidime-avibactam against β-lactamase-producing Enterobacteriaceae and Pseudomonas aeruginosa. Antimicrobial agents and chemotherapy 2014: AAC. 02894-14). Briefly, two-fold dilutions of each meropenem/BP or meropenem/TG from 0.015 to 16 μg/mL and 1 to 64 μg/mL respectively were made in cation adjusted Mueller-Hinton Broth (CAMHB) in a 96-well microtitre plate using the checkerboard method (King et al). A 0.5 McFarland-standardized bacterial inoculum was added to make a total volume of 100 μl in each microtitre well. The initial inoculum density was determined by plating out MHA plates for MBC testing. The plates were then incubated for 18-22 h at 37° C. under aerobic conditions. The MIC was determined as the lowest concentration at which there was no visible growth. Control wells included the amount of solvent used in dissolving drug candidates.

### Serum Effects in the MIC

The effect of serum on the MIC of combination was performed similar to the MIC method described above however, in this case 100% human serum was added to the broth to make up 50% human serum in the final broth. Two CRE strains were used for this experiment (E. coli NDM-1 and K. pneumoniae IMP-8).

### Time-Kill Assays

Time-kill studies were performed according to previously published methods, including those described by CLSI document M26-A. E. coli NDM-1, K. pneumoniae IMP-8 and K. pneumoniae NDM were the strains selected for this assay. Briefly, freshly prepared colonies were re-suspended in 10 mL CAMHB and incubated in a shaking incubator (37° C., 180 rpm) for 1 to 2 h. Cultures were then diluted to a 0.5 McFarland standard (approximately 10⁸ CFU/mL) and further diluted 1:20 in CAMHB so that the starting inoculum was approximately 5×10⁶ CFU/mL. Meropenem was added to the prepared bacterial suspensions so that the final meropenem concentration was 2×, 4× or 8× the MIC of meropenem-BP, and BP was added to a final concentration of 64 μg/mL. A growth control with no antibiotic was also included. The starting inoculum was determined from the growth control tube immediately after dilution and was recorded as the count at time zero. After addition of antibiotics, the starting inoculum was approximately 1×10⁶ to 5×10⁶ CFU/mL. Tubes were incubated in a shaking incubator at 37° C., 180 rpm, and viability counts were performed at 1, 2, 4, 6, 8 and 24 h by removing 100 μL of the culture, diluting as appropriate, and plating 100 μL on MHA. MHA plates were incubated at 37° C. for at least 18 h. Colonies were counted, and the results were recorded as the number of CFU/mL. A ≥3−log₁₀ decrease in the number of CFU/mL was considered bactericidal.

### Cell Culture

Human hepatoma (HepG2) cells were cultured in Dulbecco minimum essential medium (Lonza Biowhittaker, Switzerland) supplemented with 1% pen/strep/fungizone, 1% L-glutamine and 10% fetal bovine serum. The cells were maintained under the atmosphere of 5% CO₂ at 37° C. Once 80% confluence had been reached, cells were enzymatically detached (trypsin) and utilized for the cell viability assay.

### Cell Viability Assay

The effect of MPR-SMN-05 on cell viability was determined using the 3-(4,5-Dimethyl-2-thiazolyl)-2,5-diphenyl-2H-tetrazolium bromide (MTT) assay according to previously reported studies by Mosmann (Mosmann T. Rapid colorimetric assay for cellular growth and survival: application to proliferation and cytotoxicity assays. Journal of immunological methods 1983; 65: 55-63). Approximately 15, 000 cells were seeded in a 96-well microtiter plate and allowed to adhere overnight. The cells were subsequently exposed to a range of 0 to 250 mg/L MPR-SMN-05 (experiments were done in triplicate). After incubation with MPR-SMN-05 for 24 h, cells were washed twice with 0.1 M phosphate buffer saline (PBS) and incubated with 20 μl MTT salt solution (5 mg/ml in 0.1 M PBS) and 100 μl complete culture medium (CCM) (37° C., 4 h). The MTT salt solution was then discarded and 100 ml of DMSO was added to each well and incubated (37° C., 1 h) to dissolve the formazan crystals. The optical density was measured at 570/690 nm employing a spectrophotometer (Bio-Tek uQuant, Winooski, VT). The results were expressed as percentage cell viability vs. log concentration of MPR-SMN-05, from which the half maximal inhibitory concentration (IC₅₀) was determined. Control treatments were represented by an equal amount of DMSO solvent in CCM without the test compound.

### Cytotoxicity Assay

The lactate dehydrogenase (LDH) cytotoxicity detection kit (Roche, Mannheim, Germany) was used to measure the cell damage and cytotoxicity in MPR-SMN-05-treated HepG2 cells. Extracellular LDH activity was quantified by incubating 100 ml of cell culture supernatant with 100 ml substrate solution (diaphorase/NAD+; INT/sodium lactate) in a 96 well microtiter plate at room temperature for 30 min. The assay was performed in triplicate. Optical density (OD) was measured following incubation at 500 nm using a spectrophotometric plate reader (Bio-Tek uQuant, Winooski, VT).

### Statistical Analysis

All the assays were achieved at least in duplicates. Results are provided as mean±standard deviation. The statistical analyses of the experiments were performed using the statistic One-way Anova test. A p value of <0.05 was considered statistically significant.

### Bio-Analytical Methods for Determination of Meropenem, BP1 and Meropenem-BP1 Combination Concentrations in Mice Plasma

### Instrumentation

The liquid chromatography tandem mass spectrometry (LC-MS) system consisted of an Agilent Series 1100, with an online degasser, gradient pump and an auto-sampler coupled to a time-of-flight mass spectrometer analyser (TOF-MS) maXis 4G electrospray ionization (ESI) instrument (Bruker Daltonics, Bremen, Germany). All results were stored and analysed with Data Analysis 4.0 SP 5 (Bruker Daltonics).

### Preparation of Standards and Calibration Curves

Individual stock solutions of meropenem, BP-1 and internal standard (IS) TG-1 were prepared by dissolving 10 mg of each substance in 10 mL of 50% methanol in distilled water. The solutions were stored at refrigerated temperature (0 to 4° C.). Calibration standards were prepared by spiking working standard solutions and IS into 100 μL of blank plasma of untreated mice to yield the analytes concentrations of 25, 50, 100, 200, 400, 800 and 1000 ng/mL in plasma for both compounds. 200 ng/mL of the IS was used throughout the experiments for both analytes and samples. Quality control (QC) samples at lower limit of quantification (LLQC), low (LQC), middle (MQC) and high (HQC) concentrations (10; 75, 500 and 900 ng/mL respectively) were prepared separately in the same manner.

### Chromatographic Conditions

A YMC Triart C₁₈ column (YMC Europe Gmbh, Dislanken, Germany), with spherical hybrid silica particles (150 mm×3.0 mm I.D. S-3 μm) equipped with the corresponding guard column was used for HPLC separation. Two mobile phases, namely 0.1% FA in water and 0.1% FA in ACN were used. The flow rate was 0.3 mL min-1 and the temperature of the column oven was set at 25° C. The gradient profile was initially from 25% to 50% ACN in 10 min, then in 2 min reached 75%, after which time the mobile phase was returned to the initial conditions (25% ACN) in 3 min and held for equilibration for 5 min. The sample injection volume was 5 μL.

### Mass Spectrometric Analysis

A Bruker Daltonics instrument, maXis 4G ESI time of flight mass spectrometry (TOF-MS) was used to obtain accurate mass spectra of target analytes. The optimized MS conditions and acquisition parameters were as follow: source type, ESI; ion polarity, positive; nebulizer, 1.5 bar; capillary, 6000 V; dry heater, 180° C.; scan range, m/z 300-1200; end plate offset, −500 V; dry gas, 8.0 L/min; collision cell radiofrequency, 3000 Vpp, collision energy, 7 eV. These conditions have been applied for both of the target compounds (meropenem and BP1). However, different MS/MS conditions were optimised for each analyte as follow: Isolation masses were m/z; isolation width was set respectively at 38, 25; isolation energy 21, 24; and acquisition factor 2, 2.

### Plasma Samples

Blank plasma samples were obtained from Life Technologies (Burlington, ON, Canada) and kept at −20° C. prior to analysis.

Blood samples were collected by cardiac puncture from mice after post-dosing with analytes, centrifuged at 3500 rpm for 10 minutes, plasma was separated and stored at −80° C. before bioanalysis. Before any experiment, samples were defrosted at room temperature.

### Sample Preparation

Aliquots of 100 μL of blank plasma, calibration standards, QC samples, with IS were topped up to 1 mL with methanol to induce the precipitation of plasma proteins. Treated mice plasma samples followed the same procedure. The mixture was vigorously mixed for 30 seconds, followed by centrifugation at 15000 rpm, 4° C. for 10 min. The supernatants were filtered through a DSC-18-SPE cartridge (50 mg/mL). The filtered samples (500 μL) were transferred into auto-sampler vials for injection into the chromatographic system. All thawing of frozen plasma samples were completed at room temperature.

### Stability

Stabilities of BP1 in plasma was estimated by assay of three replicates of QC samples at low, medium, and high concentrations under different conditions: short-term stability after storing at room temperature (25° C.) for 6 hours; freeze/thaw stability through three freeze/thaw cycles (−80° C. to 25° C.). The post-preparative stability was examined after 24 hours in the auto-sampler maintained at 25° C. The stock solution stabilities of BP1 were determined after storage at 4° C. The quantified concentrations of stabilities were compared to the theoretical concentrations.

### Results

### MICs

To investigate the potential activity of BP1 and TG1 as MBLs inhibitors well characterized carbapenem-resistant bacteria expressing known class B carbapenemases were used. MICs using the broth micro-dilution method was conducted according to CLSI guidelines (Clinical and Laboratory Standards Institute. Performance Standards for Antimicrobial Susceptibility Testing: Twenty-Fourth Informational Supplement M100-S24. CLSI, Wayne, PA, USA, 2014) using cation adjusted Mueller-Hinton broth for meropenem, alone (See table 4) and in combination with the metal chelating agents BP1 and TG1 at variable concentrations (two-fold dilutions from 1 to 64 μg/mL). Escherichia coli ATCC 25922 was used as the control.

BP1 and TG1 were able to restore the activities of the carbapenem antibiotic (meropenem) against the class of MBL-producing bacteria at concentrations as low as 0.03 mg/L. None of the serine β-lactamases types were susceptible to the combinations of the carbapenem/metal chelating agents, which substantiate the mode of action of these compounds.

### Serum Effect

Serum had no significant effect on the MIC of meropenem/BP1 combinations as the values still fall within the susceptible range and only vary by ±1 to 2 folds dilution for both of the two isolates investigated (E coli NDM-1 and K pneumoniae IMP-8). This variation is accepted according to CLSI guidelines as one repeats the same experiment.

### Time-Kill Kinetics

Time-kill study for meropenem/BP1 was performed on E. coli NDM-1 and K. pneumoniae IMP-8. The combined carbapenem and MBL inhibitor caused decreases in the number of CFU/mL relative to the initial bacterial density over the increasing time points (0, 1, 2, 4, 6, 8 and 24 h) against both strains at all the different concentrations of the MIC that has been tested (2×, 4×, 8×MIC) (see FIG. 1). A 3 log₁₀ decrease in the number of CFU/ml was observed when treated with meropenem/BP1 at 4× and 8×MICs at 4 h. At 2×MIC 3 log₁₀ decrease was seen at 6 h after treatment with the combined molecules.

The time-kill kinetics results indicate that the combination of the MBL inhibitor (BP1) and the carbapenem antibiotic (meropenem) has a bactericidal activity against the tested strains of carbapenem-resistant Enterobacteriaceae.

Combination therapy of selected BP compounds with meropenem was compared with monotherapy of meropenem alone. An analysis of the results is given in Table 1 and Table 2.

TABLE 1
Comparative analyses of combination therapy versus monotherapy

| Compound | P value | R squared |
|---|---|---|
| BP 1 + Meropenem | 0.0484 | 0.3357 |
| BP 6 + Meropenem | 0.0441 | 0.3464 |
| BP 10 + Meropenem | 0.0347 | 0.3738 |
| BP 14 + Meropenem | 0.04 | 0.3577 |

Analyses were conducted on the lowest dose administered, 32 mg/L BP+0.5 mg/L meropenem

TABLE 2
Statistical analysis of the variation exhibited by combination
therapy versus monotherapy and no therapy

| Parameter | BP + Meropenem* |
|---|---|
| Mean square | 98.4 |
| F ratio | 17.14 |
| % of total variation | 77.19 |
| P value | 0.0013 |

*Represents the mean log₁₀ cfu/ml for all BP compounds per time point.
There was great similarity (P = 0.9986) amongst the performance of the BP compounds
Analyses were conducted on the lowest dose administered, 32 mg/L BP + 0.5 mg/L meropenem

### Cell Viability

Cell viability assay results are shown in Table 3.

TABLE 3
Half the maximal inhibitory concentrations of selected metallo-
beta-lactamase inhibitors (MBLI's) on HepG2 cells

| MBLI | IC₅₀ (μg/ml) |
|---|---|
| BP 1 | 59.96 |
| BP 6 | 51.83 |
| BP 10 | 61.98 |
| BP 14 | 42.34 |

### Cytotoxicity

The compound BP1 was safe at the active concentrations demonstrated by the in vitro studies. The effect on cell lines was only visible at high concentrations which were much higher than the MIC reported in this study.

TABLE 4
MICs in μg/mL and in μM of combinations of carbapenem antibiotic (meropenem),
beta-lactam metallo-beta-lactamase inhibitor (BP1) and metal chelating agents (TG1).

| | | | | | μg/mL-μM | μg/mL-μM | ug/mL-uM |
|---|---|---|---|---|---|---|---|
| Bacterial Strain | μg/mL | μM | μg/mL | μM | MEM | TG1 | BP1 |
| (CRE) | MEM/TG1* | MEM/TG1 | MEM/BP1** | MEM/BP1 | alone*** | alone**** | alone***** |
| E. coli IMP-1 | 0.03/32 | 0.08/65.5 | 0.06/32 | 0.16/42 | 16-42 | >64-131 | >64-84 |
| | 0.03/64 | 0.08/131 | 0.06/64 | 0.16/84 | | | |
| E. cloacae VIM-1 | 0.25/32 | 0.66/65.5 | 0.25/32 | 0.66/42 | 8-21 | >64-131 | >64-84 |
| | 0.125/64 | 0.33/131 | 0.25/64 | 0.66/84 | | | |
| E. coli NDM-1 | 0.25/16 | 0.66/32.75 | 1/16 | 2.62/21 | >16-42 | >64-131 | >64-84 |
| | 0.06/32 | 0.16/65.5 | 0.25/32 | 0.66/42 | | | |
| E. coli NDM-4 | 0.125/16 | 0.33/32.75 | 2/16 | 5.25/21 | >16-42 | >64-131 | >64-84 |
| | 0.06/32 | 0.16/65.5 | 1/32 | 2.62/42 | | | |
| E. cloacae IMP-8 | 0.125/16 | 0.32/32.75 | 1/16 | 2.62/21 | 8-21 | >64-131 | >64-84 |
| | 0.06/32 | 0.16/65.5 | 0.125/32 | 0.32/42 | | | |
| K. pneumoniae IMP-8 | 0.125/32 | 0.33/65.5 | 0.25/32 | 0.66/42 | 8-21 | >64-131 | >64-84 |
| | 0.06/64 | 0.16/131 | 0.125/64 | 0.33/84 | | | |
| K. pneumonia VIM-1 | 0.25/16 | 0.66/32.75 | 1/16 | 2.62/21 | >16-42 | >64-131 | >64-84 |
| | 0.25/32 | 0.66/65.5 | 0.5/32 | 1.31/42 | | | |
| K. pneumonia IMP-1 | 2/32 | 5.25/65.5 | 2/32 | 5.25/42 | >16-42 | >64-131 | >64-84 |
| | 0.5/64 | 1.31/131 | 1/64 | 2.62/84 | | | |
| P. rettgeri NDM-1 | 1/32 | 2.62/65.5 | 2/32 | 5.25/42 | >16-42 | >64-131 | >64-84 |
| | 0.5/64 | 1.31/131 | 1/64 | 2.62/84 | | | |

*Meropenem in combination with a metal chelator (TG1)
**Meropenem in combination with a beta-lactam MBL inhibitor (BP1)
***Meropenem alone
****Metal chelator (TG1) alone
*****Beta-lactam MBL inhibitor (BP1) alone

### In Vivo Pharmacokinetics

This study was approved by the institutional Animal Research Ethics Committee of the University of KwaZulu-Natal (approval reference AREC/013/016D). Female Balb/c (6-8 weeks old) were purchased from the Biomedical Resource Unit (BRU). Experimental animals received a combination of Meropenem (10 mg/kg·b·w) and BP1 (10 mg/kg·b·w) via i.p. administration. Animals where then sacrificed periodically at 0, 5, 15, 30, 45, 60, 90 and 120 min post dose, in order to determine plasma pharmacokinetics of meropenem and BP1, respectively. Plasma was collected via cardiac puncture and stored at −80° C.

### LC-MS Quantification

An Agilent series 1100 (Agilent Technologies, Waldbronn Germany) liquid chromatography system was coupled to a maXis 4G quadrupole-time-of-flight mass spectrometry (Q-TOF-MS) instrument (Bruker Daltonics, Bremen, Germany).

Chromatographic separation was achieved using an Ascentis® Express F5 column (5 cm×2.1 mm, 2.7 μm particle size) with a gradient mobile phase comprised of Millipore water (0.1% v/v formic acid) (A) and Acetonitrile (0.1% v/v formic acid) (B). The gradient method started from 5.0 to 95.0% B in 8.0 minutes, then held at 95% for 2.0 minutes and thereafter it was brought back to 5% over 1 minute. The column equilibration time was 4 minutes with a flow rate of 0.4 mL min⁻¹ and the column oven temperature at 25° C. The injection volume was 10 μL and the total run time of the method was 15 minutes.

Quantitative and qualitative studies were conducted using MS mode via an ESI interface, with the following source parameters: end plate offset −500V; capillary 5 kV; nitrogen nebulizer gas 1.5 bar; dry gas 8.0 L min-1 and a drying temperature of 200° C. The molecular ions optimized were m/z 325.1 for BP1, m/z 384.2 for Meropenem and m/z 350.1 for Ampicillin (IS). Results were analyzed using Data Analysis 4.0 SP 5 (Bruker Daltonics). All data are expressed as a mean±SD.

### In Vivo Infection Model

This study was approved by the institutional Animal Research Ethics Committee of the University of KwaZulu-Natal (approval reference AREC/081/015D). Female Balb/c (6-8 weeks old) were purchased from the Biomedical Resource Unit (BRU).

All animal experiments were approved by the Institutional Animal Research Ethics Committee of the University of KwaZulu-Natal (approval number AREC/081/015D). A thigh infection protocol was performed as described by Michail et al (2013). Briefly, six-week old, pathogen free-specific, male Bagg inbred albino c-strain (BALB/c) mice weighing 20-25 g (n=40), were rendered neutropenic (neutrophils<100/mm³) by pre-treatment with cyclophosphamide (i.p.) at 4 days (150 mg/kg) and 1 day (100 mg/kg). Infection of the left thigh was conducted by i.m. injection of a K. pneumoniae 449 bacterial suspension (100 μL) containing 107-10⁸ CFU/ml, two hours before the initiation of treatment with a combination of meropenem and BP1 (100 mg/kg·b·w each). The mice were randomly separated into two groups, the infected control and the treated group. Mice were humanely euthanized, by halothane overdose, at 2 h, 4 h, 6 h and 8 h post treatment. The left thigh muscle was then aseptically removed and homogenized in 5 ml of phosphate buffered saline (PBS). Homogenates were serially diluted and plated onto antibiotic-free Mueller-Hinton agar plates, and incubated at 35° C. for 24 h. Following the incubation period, the plates were assessed for growth and quantitatively enumerated using colony forming units (CFU), the titer was then expressed as log 10 CFU/thigh muscle.

### Results

### In Vivo Pharmacokinetics

The results are set out in FIG. 4 and in Table 5 below.

TABLE 5
Pharmacokinetics of BP1 and Meropenem in a healthy murine model.
Data is presented as a mean ± SD (n = 3)

| pK parameters | BP1 | Meropenem |
|---|---|---|
| Cₘₐₓ (ng mL⁻¹) | 1933 ± 88.2 | 1273 ± 33.4 |
| Tₘₐₓ(h) | 0.25 | 0.25 |
| Kₑₗ(h⁻¹) | 1.72 | 0.73 |
| T_{1/2}(h) | 0.40 | 0.47 |
| AUC 0-t (h ng mL⁻¹) | 1064 | 635 |

It was found that meropenem and BP1 displayed complementary pharmacokinetic properties.

### In Vivo Infection Model

The results are set out in FIGS. 5 and 6 which show changes in thigh CFU counts following treatment with BP1 over a 24-hour period (data presented as a mean±SD (n=3) and plasma BP1 concentrations over the 8-hour treatment period (data presented as a mean±SD (n=3).

Table 6 shows the minimum inhibitory concentration (MIC) of meropenem co-administered with all of the synthesized and tested metallo-β-lactamase (MBL) inhibitors of general formula (I) against two different carbapenase-producing microorganisms.

TABLE 6
MINIMUM INHIBITORY CONCENTRATION (MIC) OF MEROPENEM CO-ADMINISTERED WITH BP
COMPOUNDS AGAINST CARBAPENEMASE-PRODUCING ORGANISMS

| | | Escherichia | Klebsiella |
|---|---|---|---|
| | | coli | pneumoniae |
| Name (by | | NDM-1 | 449 |
| MICRO) | Structure | [a + b]¹ | [a + b]¹ |
| BP1 | 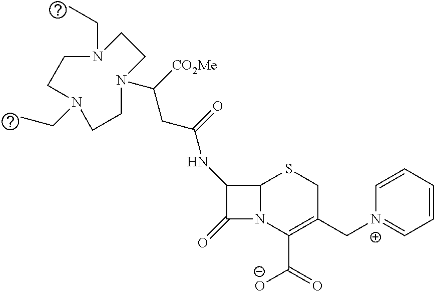 [SMILES: CCN1CCN(CC)CCN(C(CC(=O)NC2C(=O)N3C(C(=O)[O-])=C(C[n+]4ccccc4)CSC23)C(=O)OC)CC1] | 0.06 + 64 0.125 + 32 0.125 + 64 0.25 + 32 0.25 + 64 0.5 + 32 0.5 + 64 1 + 8 1 + 16 2 + 16 4 + 8 8 + 8 16 + 8 32 + 4 | 0.125 + 64 0.25 + 64 0.5 + 32 1 + 16 2 + 8 4 + 4 8 + 4 16 + 4 32 + 4 |
| BP2 | 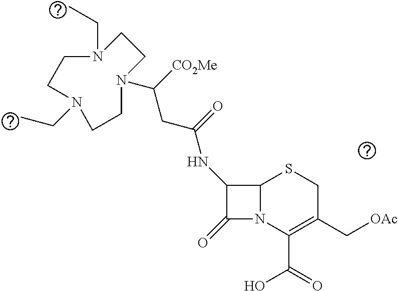 [SMILES: CCN1CCN(CC)CCN(C(CC(=O)NC2C(=O)N3C(C(=O)O)=C(COC(C)=O)CSC23)C(=O)OC)CC1] | 0.125 + 32 0.125 + 64 0.25 + 32 0.25 + 64 0.5 + 32 0.5 + 64 1 + 32 1 + 64 | 1 + 32 2 + 16 2 + 32 |
| BP3 | 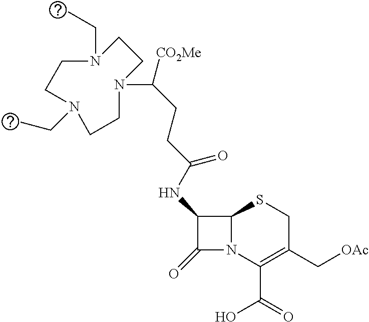 [SMILES: CCN1CCN(CC)CCN(C(CCC(=O)N[C@@H]2C(=O)N3C(C(=O)O)=C(COC(C)=O)CS[C@H]23)C(=O)OC)CC1] | 0.06 + 64 0.125 + 64 0.25 + 32 | 0.06 + 64 0.125 + 32 0.25 + 16 0.5 + 16 1 + 16 2 + 8 |
| BP4 | 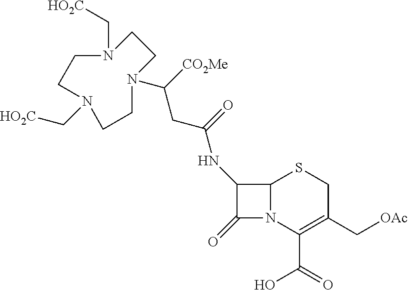 [SMILES: COC(=O)C(CC(=O)NC1C(=O)N2C(C(=O)O)=C(COC(C)=O)CSC12)N1CCN(CC(=O)O)CCN(CC(=O)O)CC1] | 0.5 + 32 0.5 + 32 1 + 16 2 + 16 4 + 8 8 + 8 16 + 8 32 + 8 | 0.25 + 64 0.5 + 32 1 + 32 2 + 8 4 + 8 8 + 8 16 + 8 32 + 8 |
| BP5 | 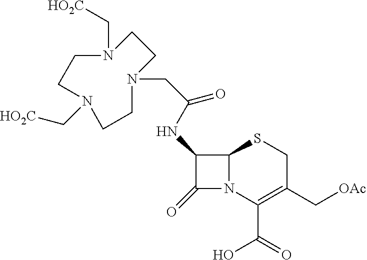 [SMILES: CC(=O)OCC1=C(C(=O)O)N2C(=O)[C@@H](NC(=O)CN3CCN(CC(=O)O)CCN(CC(=O)O)CC3)[C@H]2SC1] | 0.06 + 32 0.125 + 16 0.25 + 8 0.5 + 8 1 + 8 2 + 8 4 + 8 8 + 8 16 + 8 32 + 4 | 0.125 + 16 0.25 + 16 0.5 + 8 1 + 8 2 + 8 4 + 4 8 + 4 16 + 4 32 + 4 |
| BP6 | 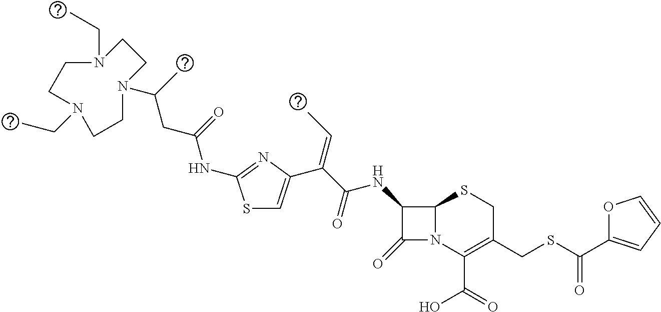 [SMILES: C/C=C(/C(=O)N[C@@H]1C(=O)N2C(C(=O)O)=C(CSC(=O)c3ccco3)CS[C@H]12)c1csc(NC(=O)CC(C)N2CCN(CC)CCN(CC)CC2)n1] | 0.06 + 8 0.125 + 8 0.25 + 8 0.5 + 8 1 + 4 2 + 4 4 + 4 8 + 4 16 + 4 32 + 2 | 0.25 + 8 0.5 + 8 1 + 4 2 + 4 4 + 4 8 + 4 16 + 4 32 + 2 |
| BP7 | 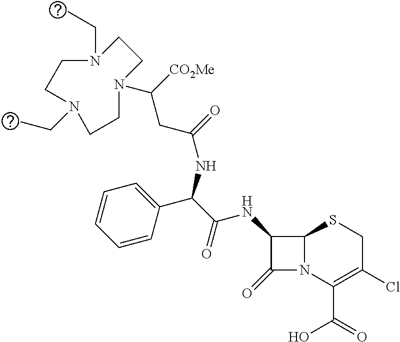 [SMILES: CCN1CCN(CC)CCN(C(CC(=O)N[C@@H](C(=O)N[C@@H]2C(=O)N3C(C(=O)O)=C(Cl)CS[C@H]23)c2ccccc2)C(=O)OC)CC1] | 0.25 + 64 0.5 + 16 1 + 8 2 + 8 4 + 8 8 + 8 16 + 4 32 + 4 | 0.125 + 64 0.25 + 32 0.5 + 16 1 + 16 2 + 8 4 + 4 8 + 4 16 + 4 32 + 4 |
| BP8 | 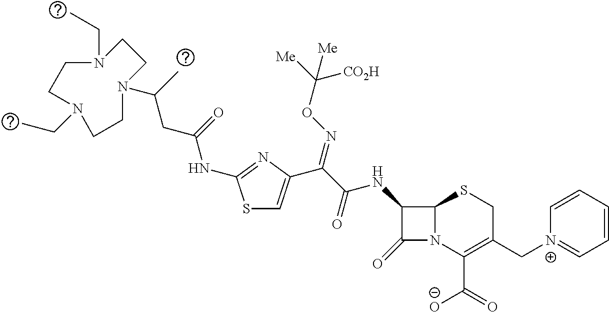 [SMILES: CCN1CCN(CC)CCN(C(C)CC(=O)Nc2nc(/C(=N\OC(C)(C)C(=O)O)C(=O)N[C@@H]3C(=O)N4C(C(=O)[O-])=C(C[n+]5ccccc5)CS[C@H]34)cs2)CC1] | 0.125 + 16 0.25 + 8 0.5 + 8 1 + 8 2 + 8 4 + 8 8 + 8 16 + 8 32 + 4 | 0.125 + 16 0.25 + 16 0.5 + 8 1 + 8 2 + 8 4 + 8 8 + 8 16 + 8 32 + 4 |
| BP9 | 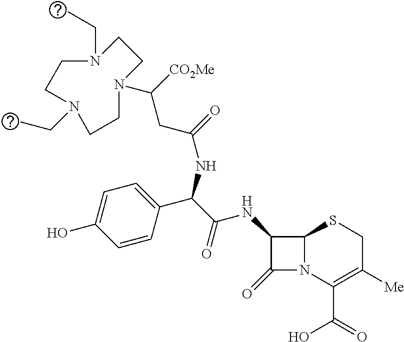 [SMILES: CCN1CCN(CC)CCN(C(CC(=O)N[C@@H](C(=O)N[C@@H]2C(=O)N3C(C(=O)O)=C(C)CS[C@H]23)c2ccc(O)cc2)C(=O)OC)CC1] | 0.06 + 32 0.125 + 16 0.25 + 16 0.5 + 8 1 + 8 2 + 4 4 + 4 8 + 4 16 + 4 32 + 2 | 0.125 + 32 0.25 + 16 0.5 + 16 1 + 8 2 + 4 4 + 4 8 + 4 16 + 4 32 + 2 |
| BP10 | 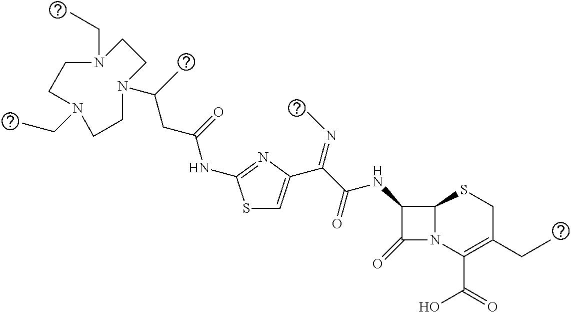 [SMILES: CCC1=C(C(=O)O)N2C(=O)[C@@H](NC(=O)/C(=N/C)c3csc(NC(=O)CC(C)N4CCN(CC)CCN(CC)CC4)n3)[C@H]2SC1] | 0.06 + 16 0.125 + 8 0.25 + 8 0.5 + 4 1 + 4 2 + 4 4 + 4 8 + 4 16 + 4 32 + 2 | 0.125 + 8 0.25 + 8 0.5 + 8 1 + 4 2 + 4 4 + 4 8 + 2 16 + 2 32 + 2 |
| BP11 | 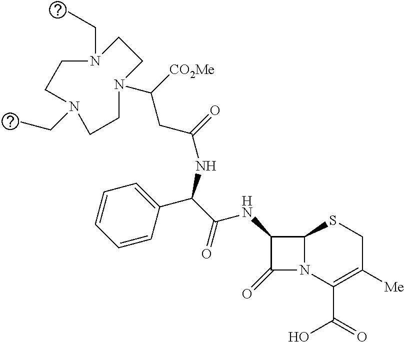 [SMILES: CCN1CCN(CC)CCN(C(CC(=O)N[C@@H](C(=O)N[C@@H]2C(=O)N3C(C(=O)O)=C(C)CS[C@H]23)c2ccccc2)C(=O)OC)CC1] | 0.06 + 32 0.125 + 32 0.25 + 32 0.5 + 16 1 + 8 2 + 8 4 + 8 8 + 4 16 + 4 32 + 4 | 0.125 + 32 0.25 + 32 0.5 + 16 1 + 8 2 + 4 4 + 4 8 + 4 16 + 2 32 + 2 |
| BP12 | 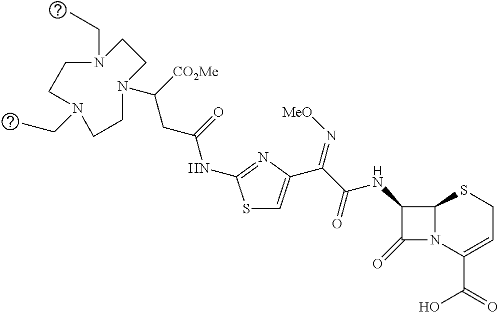 [SMILES: CCN1CCN(CC)CCN(C(CC(=O)Nc2nc(/C(=N\OC)C(=O)N[C@@H]3C(=O)N4C(C(=O)O)=CCS[C@H]34)cs2)C(=O)OC)CC1] | 0.06 + 16 0.125 + 8 0.25 + 8 0.5 + 4 1 + 4 2 + 4 4 + 4 8 + 4 16 + 4 32 + 2 | 0.06 + 32 0.125 + 8 0.25 + 8 0.5 + 4 1 + 4 2 + 4 4 + 4 8 + 2 16 + 2 32 + 2 |
| BP13 | 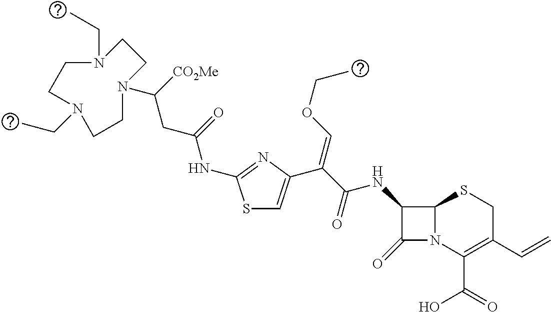 [SMILES: C=CC1=C(C(=O)O)N2C(=O)[C@@H](NC(=O)/C(=C/OCC)c3csc(NC(=O)CC(C(=O)OC)N4CCN(CC)CCN(CC)CC4)n3)[C@H]2SC1] | 0.125 + 8 0.25 + 8 0.5 + 4 1 + 4 2 + 4 4 + 4 8 + 4 16 + 4 32 + 2 | 0.25 + 8 0.5 + 8 1 + 4 2 + 4 4 + 4 8 + 4 16 + 4 32 + 2 |
| BP14 | 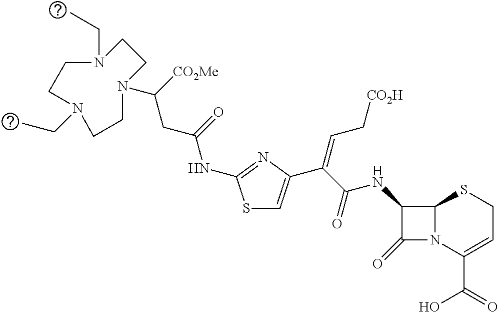 [SMILES: CCN1CCN(CC)CCN(C(CC(=O)Nc2nc(/C(=C/CC(=O)O)C(=O)N[C@@H]3C(=O)N4C(C(=O)O)=CCS[C@H]34)cs2)C(=O)OC)CC1] | 0.06 + 8 0.125 + 4 0.25 + 4 0.5 + 4 1 + 2 2 + 2 4 + 2 8 + 1 16 + 1 32 + 1 | 0.125 + 16 0.25 + 16 0.5 + 8 1 + 8 2 + 8 4 + 8 8 + 4 16 + 4 32 + 2 |
| BP15 | 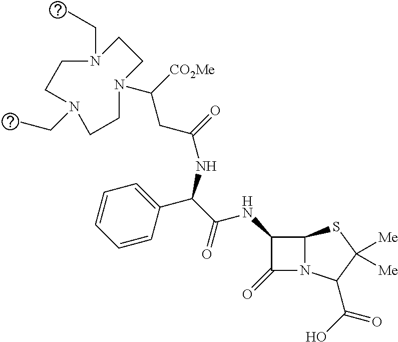 | 0.25 + 16 0.5 + 16 1 + 8 2 + 8 4 + 8 8 + 8 16 + 8 32 + 4 | 0.125 + 32 0.25 + 16 0.5 + 16 1 + 16 2 + 16 4 + 16 8 + 8 16 + 8 32 + 4 |

Minimum inhibitory concentration (MIC) for all BP's administered alone is >64 μg/ml and MIC for Meropenem administered alone is >32 μg/ml against bacterial isolates (Escherichia coli NDM-1 and Klebsiella pneumoniae 449)
Co-administration of meropenem and BPs inhibited the growth of the bacterial isolates at different concentrations
a = concentration of meropenem (μg/ml)
b = concentration of BP (μg/ml)
¹concentration of meropenem and BP that resulted in inhibition

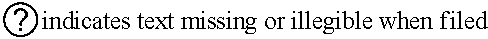

The compounds of formula (I) in combination with a β-lactam antibiotic have been found to be effective in vitro at concentrations which have no cytotoxic effect on cell lines. The bactericidal activity and effect of serum on the administered β-lactam antibiotic when used in combination with the compounds of formula (I) against metallo-β-lactamase producing Enterobacteriaceae has been determined.

MIC, times kill kinetics and serum assays were performed using the broth microdilution technique, according to the CLSI guidelines. It was found that the β-lactam antibiotic regained its activity against CRE producing MBLs where the minimum inhibitory concentration (MIC) values decreased to concentrations as low as 0.03 mcg/mL in the presence of the compounds of formula (I). The combinations have the ability to retain their bactericidal activity against resistant Enterobacteriaceae. The presence of serum also had no significant effect on the combination.

1. A metallo-R-lactamase (MBL) inhibitor of general formula (I)
C-A-L (I)
in which:
C is a functionalised aza-cycloalkane zinc chelating moiety,
L is a β-lactam moiety which may be functionalised, and
A is a C₁-C₁₀ linear or cyclic linker, which may be functionalised.

2. The inhibitor as claimed in claim 1, wherein the functionalised aza-cycloalkane zinc chelating moiety C is a functionalised aza-cyclononane moiety.

3. The inhibitor as claimed in claim 2, wherein the functionalised aza-cycloalkane zinc chelating moiety C is a substituted 1,4,7-triazacyclononane moiety.

4. The inhibitor as claimed in claim 3, wherein the substituted 1,4,7-triazacyclononane moiety is selected from the group consisting of a 1,4,7-triazacyclononane-1,4,7-triacetic acid (NOTA) moiety, a 1,4,7-triazacyclononane-1,4,7-triglutaric acid (NOTGA) moiety, a 1,4,7-triazacyclononane-1-succinic acid-4,7-diacetic acid (NODASA) moiety, a 1,4,7-triazacyclononane-1-glutaric acid-4,7-diacetic acid (NODAGA) moiety, a 1,4,7-triazacyclononane-1,4-diacetic acid-7-p-hydroxyphenyl-acetic acid (NODAPA) moiety, a 1,4,7-tris(2-pyridylmethyl)-1,4,7-triazacyclononane moiety, a 4,7-di(2-pyridylmethyl)-1,4,7-triazacyclononane-1-acetic acid, and a 1-[(5-carboxy-2-methylpyridyl)]-4,7-bis(2-methylpyridyl)-1,4,7-triazacyclononane moiety.

5. The inhibitor as claimed in claim 3 or claim 4, wherein the linker A is connected to a nitrogen atom of the substituted 1,4,7-triazacyclononane moiety.

6. The inhibitor as claimed in any of claims 1 to 5, wherein the linker A is a moiety selected from the group consisting of saturated monoacid moieties, unsaturated monoacid moieties, saturated diacid moieties, unsaturated diacid moieties, and esters of these moieties.

7. The inhibitor as claimed in claim 6, wherein the linker A is a moiety selected from the group consisting of an acetic acid moiety, a glutaric acid moiety, a maleic acid moiety, a fumaric acid moiety, a succinic acid moiety, a p-hydroxyphenyl acetic acid moiety, and esters of these moieties.

8. The inhibitor as claimed in any of claims 1 to 7, wherein the R-lactam moiety L is selected from the group consisting of cephalosporin moieties, penem moieties and carbapenem moieties.

9. The inhibitor as claimed in claim 8, wherein the β-lactam moiety L is derived from cephalosporins, penems or carbapenems selected from

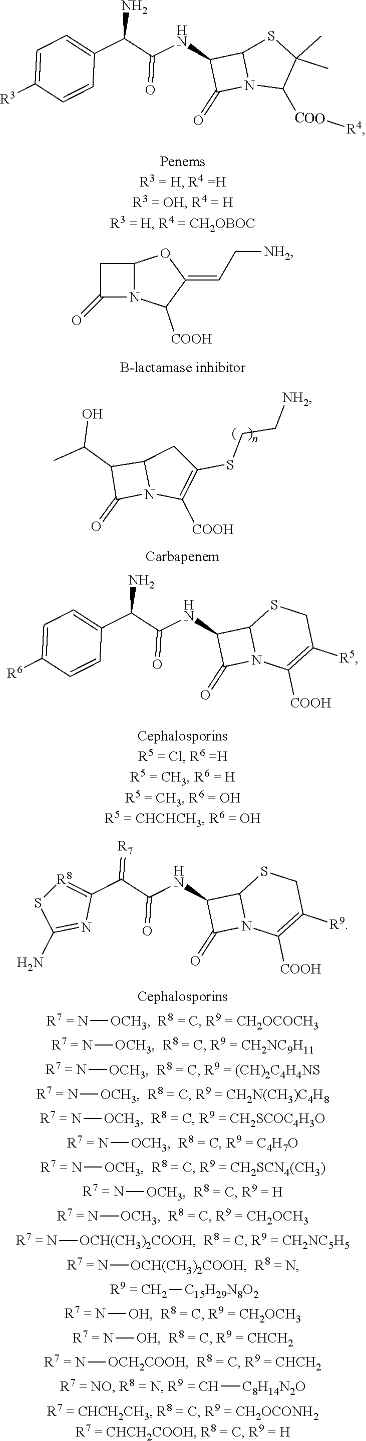

10. The inhibitor as claimed in any of claims 1 to 9, wherein the linker A is linked to the β-lactam moiety L by an amide linkage on the α position to the carbonyl of the β-lactam ring.

11. The inhibitor as claimed in claim 1, which is selected from the compounds

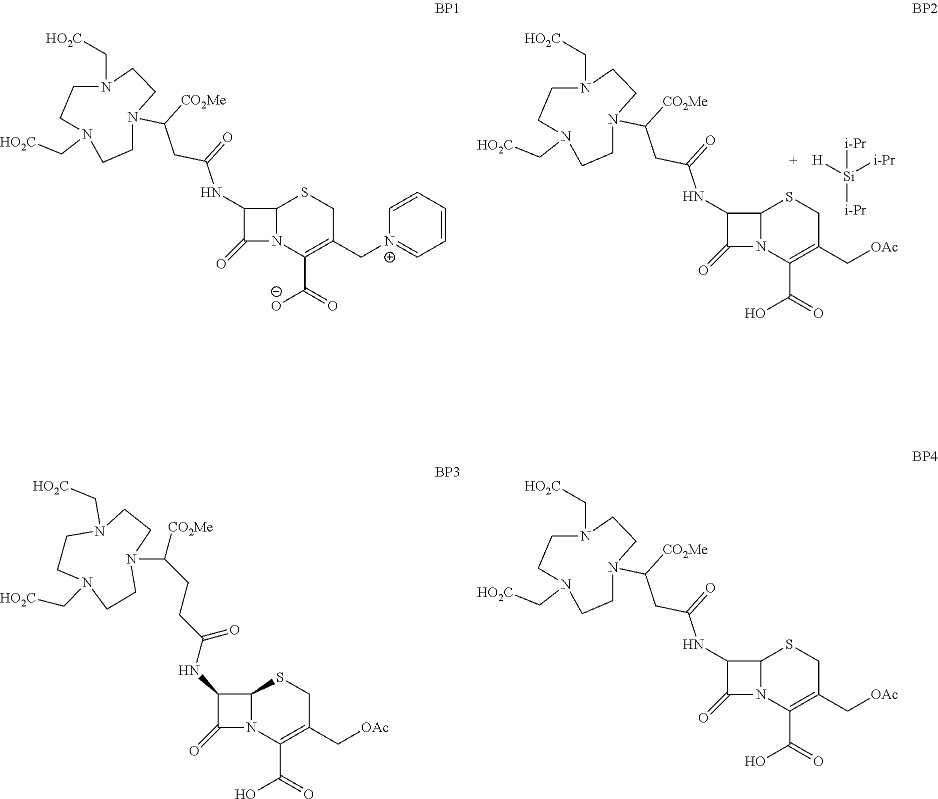

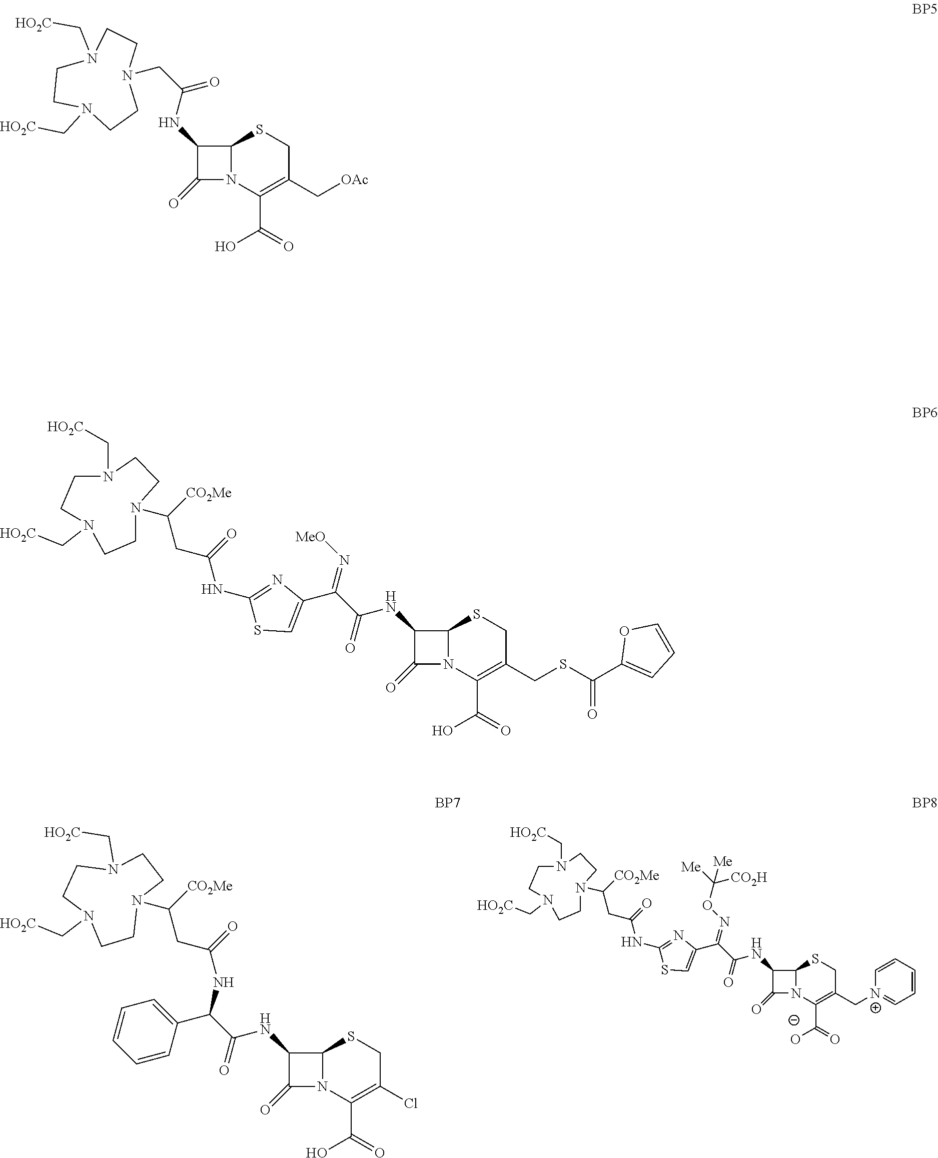 [SMILES: CC(=O)OCC1=C(C(=O)O)N2C(=O)[C@@H](NC(=O)CN3CCN(CC(=O)O)CCN(CC(=O)O)CC3)[C@H]2SC1 ; CO/N=C(/C(=O)N[C@@H]1C(=O)N2C(C(=O)O)=C(CSC(=O)c3ccco3)CS[C@H]12)c1csc(NC(=O)CC(C(=O)OC)N2CCN(CC(=O)O)CCN(CC(=O)O)CC2)n1 ; COC(=O)C(CC(=O)N[C@@H](C(=O)N[C@@H]1C(=O)N2C(C(=O)O)=C(Cl)CS[C@H]12)c1ccccc1)N1CCN(CC(=O)O)CCN(CC(=O)O)CC1 ; COC(=O)C(CC(=O)Nc1nc(/C(=N\OC(C)(C)C(=O)O)C(=O)N[C@@H]2C(=O)N3C(C(=O)[O-])=C(C[n+]4ccccc4)CS[C@H]23)cs1)N1CCN(CC(=O)O)CCN(CC(=O)O)CC1]

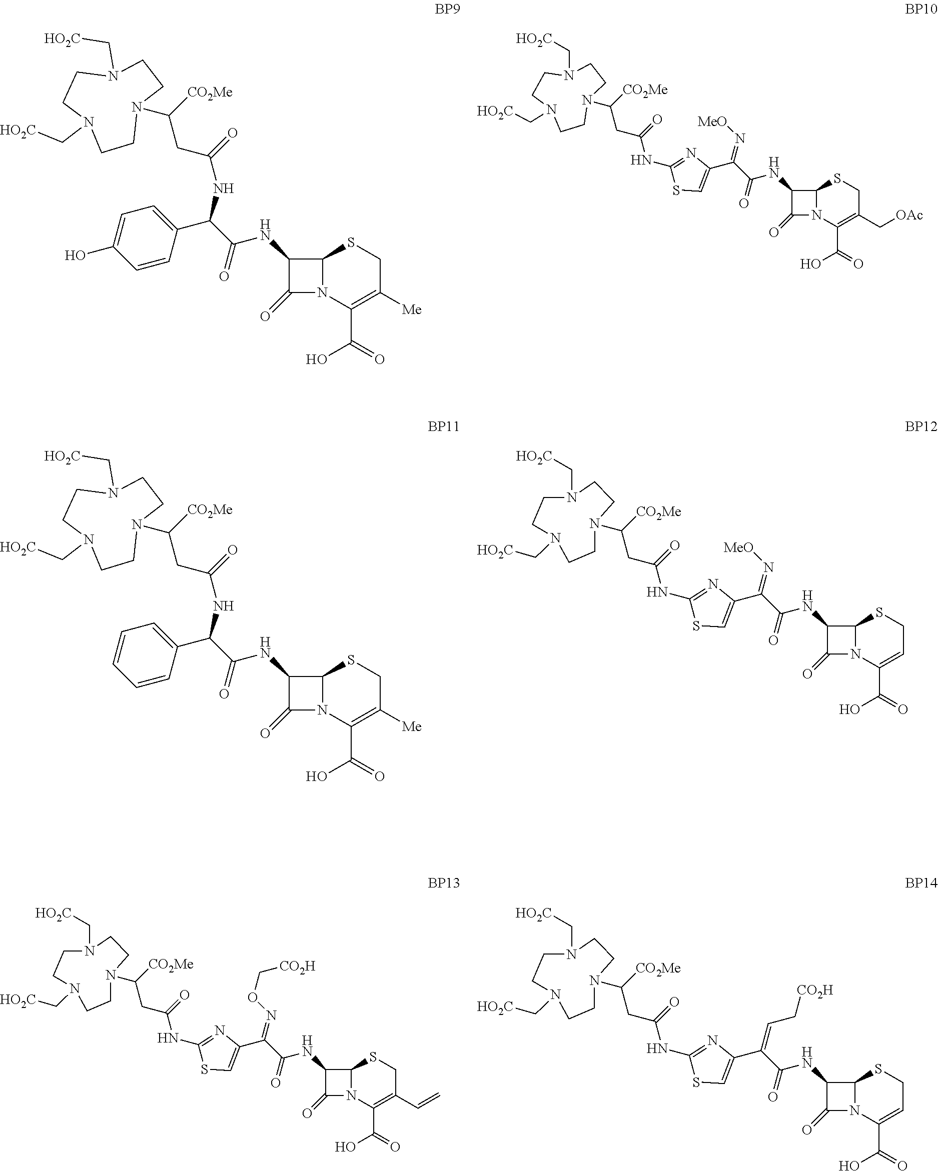

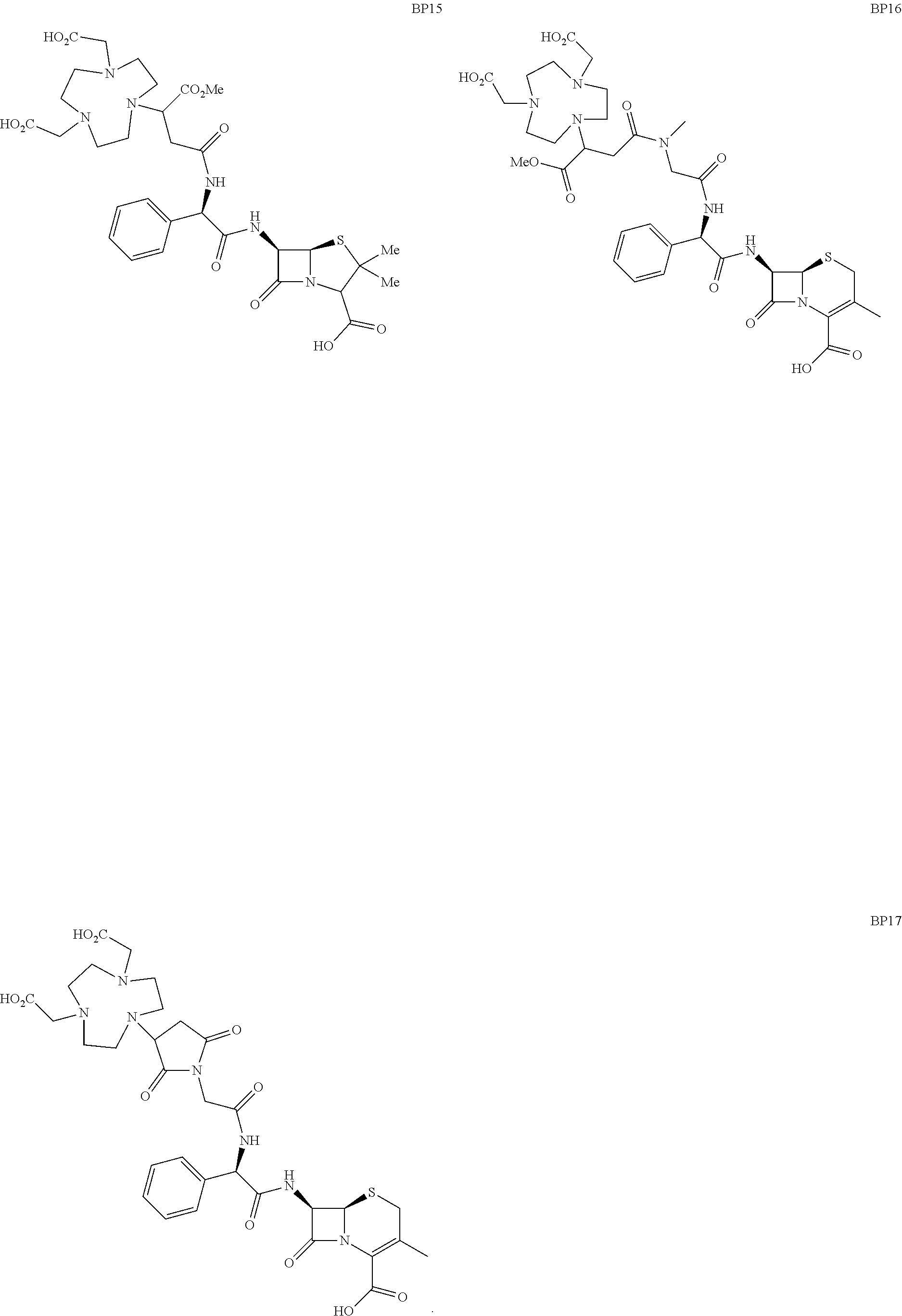

12. A pharmaceutical composition comprising a metallo-β-lactamase (MBL) inhibitor as claimed in any of claims 1 to 11 and one or more pharmaceutically acceptable carriers or excipients.

13. Use of a metallo-R-lactamase (MBL) inhibitor as claimed in any of claims 1 to 11 in the manufacture of a medicament for the treatment or prevention of a bacterial infection in a human or in a non-human mammal.

14. A metallo-R-lactamase (MBL) inhibitor as claimed in any of claims 1 to 11, or a pharmaceutical composition as claimed in claim 12, for use in a method of treating or preventing a bacterial infection in a human or in a non-human mammal.

15. The inhibitor for use or the pharmaceutical composition for use as claimed in claim 14, wherein said inhibitor or pharmaceutical composition is for administration of the metallo-β-lactamase (MBL) inhibitor simultaneously or sequentially with a β-lactam antibiotic.

16. A kit for the treatment or prevention of a bacterial infection in a human or in a non-human mammal, said kit comprising at least one of (i) a metallo-R-lactamase (MBL) inhibitor as claimed in any of claims 1 to 11 and (ii) a pharmaceutical composition as claimed in claim 12.

17. The kit as claimed in claim 16, which further includes one or more β-lactam antibiotics.

18. The kit as claimed in claim 17, wherein the one or more β-lactam antibiotics are selected from the group consisting of meropenem, imipenem, doripenem, ertapenem and combinations thereof.

19. A method of treating or preventing a bacterial infection in a human or in a non-human mammal, the method including the step of administering an effective amount of a metallo-R-lactamase (MBL) inhibitor as claimed in any of claims 1 to 11, or of a pharmaceutical composition as claimed in claim 12, to said human or to said non-human mammal.

20. The method as claimed in claim 19, which includes also administering one or more β-lactam antibiotics to said human or to said non-human mammal.

21. The method as claimed in claim 20, wherein the one or more β-lactam antibiotics are selected from the group consisting of meropenem, imipenem, doripenem, ertapenem and combinations thereof.

22. The use as claimed in claim 13, the inhibitor for use or the pharmaceutical composition for use as claimed in claim 14 or claim 15, the kit as claimed in any of claims 16 to 18, or the method as claimed in any of claims 19-21, wherein the bacterial infection is caused by a bacterial strain selected from the group consisting of E. coli IMP-1, E. coli NDM-1, E. coli VIM-1, E. cloacae VIM-1, E. cloacae IMP-1, K. pneumoniae IMP-8. K. pneumoniae VIM-1, K. pneumoniae IMP-1, K. pneumoniae NDM, and P. rettgeri NDM and combinations thereof.
